(19) United States
(12) Patent Application Publication
(10) Pub. No.: US 2005/0054582 A1
(43) Pub. Date: Mar. 10, 2005

(54) Para-amino benzoic acids as integrin antagonists

(76) Inventors: Thomas Lehmann, Wulfrath (DE); Markus Albers, Leverkusen (DE); Thomas Rolle, Leverkusen (DE); Gerhard Hessler, Hofheim (DE); Gerhard Hessler, Hofheim (DE); Masaomi Tajimi, Kyoto (JP); Karl Ziegelbauer, Haan (DE); Hiromi Okigami, Kyoto (JP); Kevin Bacon, Hyogo (JP); Haruki Hasegawa, Kyoto (JP)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/491,699

(22) PCT Filed: Sep. 20, 2002
(86) PCT No.: PCT/EP02/10563

(30) Foreign Application Priority Data
Oct. 3, 2001 (GB) 0123765.0

(51) Int. Cl. A61K 38/04; A61K 31/195; C07C 233/87

(52) U.S. Cl. 514/19; 514/562; 514/563; 562/450; 562/430

(57) ABSTRACT

The present invention relates to compounds of the general formula (I), their preparation and use as pharmaceutical compositions asintegrin antagonists, especially as α₄β and/or α₄β7 ?and/or α₉β₁ intergrin antagonists and in particular for the production of pharmaceutical compositions suitable for the inhibition or the prevention of cell adhesion and cell-adhesion mediated disorders. Examples are the treatment and the prophylaxis of atherosclerosis, asthma, chronic obstructive pulmonary disease (COPD), allergies, diabetes, inflammatory bowel disease, multiple sclerosis, myocardial ischemia, rheumatoid arthritis, transplant rejection and other inflammatory, autoimmune and immune disorders.

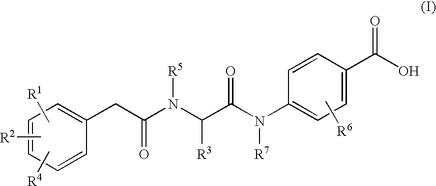

The present invention relates to compounds of formula (I), 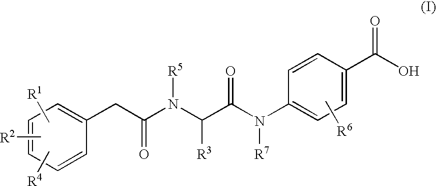
their preparation and use as pharmaceutical compositions as integrin antagonists, especially as α₄β₁ and/or α₄β₇ and/or aged integrin antagonists and in particular for the production of pharmaceutical compositions suitable for the inhibition or the prevention of cell adhesion and cell-adhesion mediated disorders. Examples are the treatment and the prophylaxis of atherosclerosis, asthma, chronic obstructive pulmonary disease (COPD), allergies, diabetes, inflammatory bowel disease, multiple sclerosis, myocardial ischemia, rheumatoid arthritis, transplant rejection and other inflammatory, autoimmune and immune disorders.

Adhesive interactions between the leukocytes and endothelial cells play a critical role in leukocyte trafficking to sites of inflammation. These events are essential for normal host defense against pathogens and repair of tissue damage, but can also contribute to the pathology of a variety of inflammatory and autoimmune disorders. Indeed, eosinophil and T cell infiltration into the tissue is known as a cardinal feature of allergic inflammation such as asthma.

The interaction of circulating leukocytes with adhesion molecules on the luminal surface of blood vessels appears to modulate leukocyte transmigration. These vascular cell adhesion molecules arrest circulating leukocytes, thereby serving as the first step in their recruitment to infected or inflamed tissue sites. Subsequently, the leukocytes reaching the extravascular space interact with connective tissue cells such as fibroblasts as well as extracellular matrix proteins such as fibronectin, laminin, and collagen. Adhesion molecules on the leukocytes and on the vascular endothelium are hence essential to leukocyte migration and attractive therapeutic targets for intervention in many inflammatory disorders.

Leukocyte recruitment to sites of inflammation occurs in a stepwise fashion beginning with leukocyte tethering to the endothelial cells lining the blood vessels. This is followed by leukocyte rolling, activation, firm adhesion, and transmigration. A number of cell adhesion molecules involved in those four recruitment steps have been identified and characterized to date. Among them, the interaction between vascular cell adhesion molecule 1 (VCAM-1) and very late antigen 4 (VLA-4, α₄β₁ integrin), as well as the interaction between mucosal addressin cell adhesion molecule 1 (MAdCAM-1) and α₄β₇ integrin, has been shown to mediate the tethering, rolling, and adhesion of lymphocytes and eosinophils, but not neutrophils, to endothelial cells under a physiologic flow condition. This suggests that the VCAM-1/VLA-4 and/or MAdCAM-1/α₄β₇ integrin mediated interactions could predominantly mediate a selective recruitment of leukocyte subpopulations in vivo. The inhibition of this interaction is a point of departure for therapeutic intervention (A. J. Wardlaw, J Allergy Clin. Immunol. 1999, 104, 917-26).

VCAM-1 is a member of immunoglobulin (Ig) superfamily and is one of the key regulators of leukocyte trafficking to sites of inflammation. VCAM-1, along with intracellular adhesion molecule 1 (ICAM-1) and E-selectin, is expressed on inflamed endothelium activated by such cytokines as interleukin 1 (IL-1) and tumor necrosis factor a (TNF-α), as well as by lipopolysaccharide (LPS), via nuclear factor κB (NF-κB) dependent pathway. However, these molecules are not expressed on resting endothelium. Cell adhesion mediated by VCAM-1 may be involved in numerous physiological and pathological processes including myogenesis, hematopoiesis, inflammatory reactions, and the development of autoimmune disorders. Integrins VLA-4 and α₄β₇ both function as leukocyte receptors for VCAM-1.

The integrin α₄β₁ is a heterodimeric protein expressed in substantial levels on all circulating leukocytes except mature neutrophils. It regulates cell migration into tissues during inflammatory responses and normal lymphocyte trafficking. VLA-4 binds to different primary sequence determinants, such as a QIDSP motif of VCAM-1 and an ILDVP sequence of the major cell type-specific adhesion site of the alternatively spliced type III connecting segment domain (CS-1) of fibronectin.

In vivo studies with neutralizing monoclonal antibodies and inhibitor peptides have demonstrated a critical role for (%4 integrins interaction in leukocyte-mediated inflammation. Blocking of VLA-4/ligand interactions, thus, holds promise for therapeutic intervention in a variety of inflammatory, autoimmune and immune diseases (Zimmerman, C.; Exp. Opin. Ther. Patents 1999, 9, 129-133).

Furthermore, compounds containing a bisarylurea moiety as a substituent were disclosed as α₄β₁ integrin receptor antagonists: WO 96/22966, WO 97/03094, WO 99/33789, WO 99/37605. However, no aminobenzoic acids or aminocycloalkylcarboxylic acids or homologues thereof or heterocyclics analogues thereof with α₄β₁ integrin receptor antagonists activity have been described.

3-[[[(phenylacetyl)amino]acetyl]amino]-benzoic acid has been described in Bio-chemistry, Vol. 26, No. 12, 1987, 3385 as a substrate for β-lactamases. N-(4-amino-phenylacetylglycyl)-4-aminophenylacetic acid has been described in J. für prakt. Chem., 4. Reihe, Band 27, 1965, 63 without giving a pharmaceutical use. N¹-[4-(eth-oxycarbonyl)phenyl]-N²-phenylacetyl)-α-glutamine and N²-benzoyl-N¹-[4-(ethoxy-carbonyl)phenyl]-α-glutamine and related compounds have been described in Minerva Medica, 58 (86), 1967, 3651 and NL 6510006 as antisecretory agents. (S)-4-[[4-carboxy-1-oxo-2-[(phenylacetyl)amino]butyl]amino]-benzeneacetic acid has been described in Drugs Exp. Clin. Res. Suppl. 1, XII, 1987, 57 as antitumor agent. N-[2-[[4-aminosulfonyl)phenyl]amino]-2-oxoethyl]-N-ethylbenzeneacetamide has been described in Eur. J. Med. Chem.-Chim. Ther. 12 (4), 1977, 387 with schistosomicide activity. N-(2-phenylacetylamino-acetylamino)-benzoic acid ethyl ester has been described in Yakugaku Zasshi 79, 1959, 1606 in decomposition studies of penicillins. Japanese publication Hei 11-269135 describes 3-aminosubstituted benzoic acid derivatives as selectin inhibitors.

None of these compounds have been described in relation to the inhibition or the prevention of cell adhesion and cell-adhesion mediated disorders.

Further to their α₄β₁ integrin antagonistic activity, the compounds of the present invention may also be used as α₄β₇ or α₉β₁ integrin antagonists.

An object of the present invention is to provide new, alternative, aminobenzoic acids or aminocycloalkylcarboxylic acids or homologues thereof or heterocyclic analogues thereof derived integrin antagonists for the treatment of inflammatory, autoimmune and immune diseases.

The present invention therefore relates to compounds of the general formula (I): 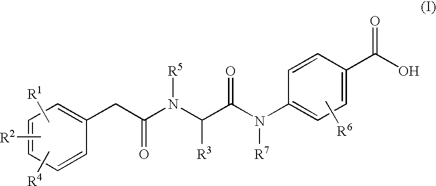
wherein
R¹ represents hydrogen, C₁-C₄-alkyl, trifluormethyl, trifluormethoxy, phenyl, —OR¹⁻², —SR¹⁻², NR¹⁻³R¹⁻⁴, —C(O)R¹⁻², S(O)R¹⁻², —SO₂R¹⁻², —CO₂R¹⁻², —OC(O)R¹⁻², —C(O)NR¹⁻³R¹⁻⁴, —NR¹⁻²C(O)R¹⁻², —SO₂NR¹⁻³R¹⁻⁴, —NR¹⁻²SO₂R¹⁻², —NR₁₋₂C(O)NR¹⁻³R¹⁻⁴-NR¹⁻²C(O)OR¹⁻⁴, —OC(O)NR¹⁻³R¹⁻⁴, halogen, cyano, nitro or amino,
wherein R¹⁻² represents hydrogen or C₁-C₄-alkyl,
wherein R¹⁻³ represents hydrogen or C₁-C₄-alkyl,
R¹⁻⁴ represents hydrogen, C₁-C₄-alkyl, C₃-C₆-cycloalkyl, C₆- or C₁₀-aryl, heteroaryl or a heterocycle,
wherein R¹⁻⁴ can optionally be substituted by 1 to 2 substituents selected from the group C₁-C₄-alkyl, phenyl, C₃-C₇-cycloalkyl, C₁-C₄-alkyloxy, halogen, nitro, cyano,
R² represents hydrogen or halogen, or
R¹ and R² together form a 4-7-membered ring, which includes the carbon atoms to which R¹ and R² are bonded and which contains up to 2 additional heteroatoms selected from the group oxygen, nitrogen or sulfur and which contains up to 2 double bonds,
   wherein the ring formed by R¹ and R² can optionally be substituted by —NH—C₆— or C₁₀-aryl, —NH-heterocyclyl or —NH-heteroaryl,
   wherein C₆- or C₁₀-aryl can optionally be substituted by 1 to 2 substituents halogen, C₁-C₄-alkyl or C₁-C₄-alkoxy,
R³ represents hydrogen, C₁-C₁₀-alkyl, C₂-C₁₀-alkenyl, C₂-C₁₀-alkynyl, —(CH₂)ₘ—C₆— or C₁₀-aryl, —(CH₂)ₘ—C₃-C₇-cycloalkyl, —(CH₂)ₘ-heterocyclyl or —(CH₂)ₘ-heteroaryl,
wherein m represents an integer of zero to six,
wherein R³ can optionally be substituted by 1 to 3 radicals R³⁻¹,
wherein R³⁻¹ represents trifluormethyl, trifluormethoxy, —OR³⁻², —NR³⁻³R³⁻⁴, —C(O)R³⁻², halogen, cyano, nitro, oxo, C₆- or C₁₀-aryl, heterocyclyl, hetero-aryl,
wherein R³⁻² represents hydrogen, C₁-C₄-alkyl, C₃-C₆cycloalkyl, C₆- or C₁₀-aryl,
and wherein R³⁻³ and R³⁻⁴ are identical or different and represent hydrogen or C₁-C₄-alkyl,
R⁴ represents hydrogen, halogen, C₁-C₄-alkyl, C₁-C₄-alkoxy, cyano, amino or nitro,
R⁵ represents hydrogen, C₁-C₁₀-alkyl, C₂-C₁₀-alkenyl, C₂-C₁₀-alkynyl, —(CH₂)ₙ—C₆— or C₁₀-aryl, —(CH₂)ₙ—C₃-C₇-cycloalkyl, —(CH₂)ₙ-heterocyclyl, —(CH₂)ₙ-heteroaryl,
wherein n represents an integer of zero to six,
wherein R⁵ can optionally be substituted by 1 to 3 radicals R⁵⁻¹,
wherein R⁵⁻¹ represents C₁-C₄ alkyl, trifluormethyl, trifluormethoxy, —OR⁵⁻², —NR⁵⁻³R⁵⁻⁴, —C(O)R⁵⁻², halogen, cyano, nitro, oxo, C₆- or C₁₀-aryl, heterocyclyl, heteroaryl,
wherein R⁵⁻² represents hydrogen, C₁-C₄-alkyl, C₃-C₆-cycloalkyl, C₆- or C₁₀-aryl or halogenated C₆- or C₁₀-aryl,
and wherein R⁵⁻³ and R⁵⁻⁴ are identical or different and represent hydrogen or C₁-C₄-alkyl, or
R³ and R⁵ together form a 4-7-membered heterocyclic ring, which includes the nitrogen atom to which R⁵ is bonded and the carbon atom to which R³ is bonded and which contains up to 2 additional heteroatoms selected from the group oxygen, nitrogen or sulfur and which contains up to 2 double bonds,
R⁶ represents hydrogen, C₁-C₄ alkyl, —OR⁶⁻¹, —NR⁶⁻²R⁶⁻³, —C(O)R⁶⁻¹, C₆-aryl, heterocyclyl, heteroaryl, halogen, cyano, nitro, hydroxy, amino, trifluoromethyl, trifluoromethoxy,
wherein R⁶⁻¹ represents hydrogen, C₁-C₄-alkyl, C₃-C₆-cycloalkyl or C₆-aryl,
wherein R⁶⁻² and R⁶⁻³ are identical or different and represent hydrogen, C₁-C₄-alkyl, C₃-C₆-cycloalkyl or C₆-aryl,
and wherein R⁶, R⁶⁻¹, R⁶⁻² and R⁶⁻³ can optionally be substituted by 1 to 2 radicals R⁶⁻⁴,
wherein R⁶⁻⁴ represents trifluoromethyl, trifluoromethoxy, halogen, cyano, nitro, hydroxy, amino and oxo
R⁷ represents hydrogen or C₁-C₄ alkyl,
or R⁷ and R³ together with the carbon atoms to which they are bonded form a cycloalkyl ring,
X represents oxygen or two hydrogen atoms,
and pharmaceutically acceptable salts thereof.

In the context of the present invention alkyl stands for a straight-chain or branched alkyl residue, such as methyl, ethyl, n-propyl, iso-propyl, n-pentyl. If not stated otherwise, preferred is C₁-C₁₀-alkyl, very preferred is C₁-C₆-alkyl, especially C₁-C₄-alkyl.

Alkenyl and alkynyl stand for straight-chain or branched residues containing one or more double or triple bonds, e.g. vinyl, allyl, isopropinyl, ethinyl. If not stated otherwise, preferred is C₁-C₁₀ alkenyl or alkinyl, very preferred is C₁-C₆ alkenyl or alkinyl.

Cycloalkyl stands for a cyclic alkyl group such as cyclopropyl, cyclobutyl, cyclo-pentyl, cyclohexyl or cycloheptyl. Preferred is C₃-C₇-cycloalkyl, especially C₅-C₆-cycloalkyl.

—(CH₂)ₘ— or —(CH₂)ₙ— represent alkandiyl chains of the length m or n. —(CH₂)ₙ—C₆— or C₁₀-aryl, —(CH₂)ₙ—C₃-C₇-cycloalkyl, —(CH₂)ₙ-heterocyclyl, —(CH₂)ₙ-heteroaryl represent the respective rings, which are bonded via the alkandiyl chain.

Halogen in the context of the present invention stands for fluorine, chlorine, bromine or iodine. If not specified otherwise, chlorine or fluorine are preferred. Halogenated stands for a substitution with 1 or 2 fluorine or chlorine atoms.

Heteroaryl stands for a monocyclic heteroaromatic system containing 4 to 9, especially 5 or 6 ring atoms, which contains 1, 2 or 3 heteroatoms independently selected from the group consisting of nitrogen, oxygen and sulfur, and which can be attached via a carbon atom or eventually via a nitrogen atom within the ring, for example, furan-2-yl, furan-3-yl, pyrrol-1-yl, pyrrol-2-yl, pyrrol-3-yl, thienyl, thiazolyl, oxazolyl, imidazolyl, triazolyl, tetrazolyl, pyridyl, pyrimidyl or pyridazinyl. C₄-C₉ heteroaryl also stands for a 4 to 9-membered ring, wherein one or more of the carbon atoms are replaced by heteroatoms. If not specified otherwise, pyridyl or thienyl are preferred.

A saturated or unsaturated heterocyclic residue (heterocycle) stands for a mono-cyclic system containing 4 to 9, especially 5 or 6 ring atoms, which contains 1, 2 or 3 heteroatoms independently selected from the group consisting of nitrogen, oxygen and sulfur, and which can contain one or more double bonds and which can be attached via a ring carbon atom or eventually via a nitrogen atom, e.g. tetrahydrofur-2-yl, pyrrolidine-1-yl, piperidine-1-yl, piperidine-2-yl, piperidine-3-yl, piperidine-4-yl, piperazine-1-yl, piperazine-2-yl morpholine-1-yl, 1,4-diazepine-1-yl or 1,4-dihydropyridine-1-yl.

If not specified otherwise, in the context of the present invention heteroatom stands preferably for O, S, N or P.

Surprisingly, the compounds of the present invention show good integrin antagonistic activity. They are therefore suitable especially as α₄β₁ and/or α₄α₇ and/or α₉β₁ integrin antagonists and in particular for the production of pharmaceutical compositions for the inhibition or the prevention of cell adhesion and cell-adhesion mediated disorders. Examples are the treatment and the prophylaxis of atherosclerosis, asthma, chronic obstructive pulmonary disease (COPD), allergies, diabetes, inflammatory bowel disease, multiple sclerosis, myocardial ischemia, rheumatoid arthritis, transplant rejection and other inflammatory, autoimmune and immune disorders.

The integrin antagonists of the invention are useful not only for treatment of the physiological conditions discussed above, but are also useful in such activities as purification of integrins and testing for activity.

In a preferred embodiment, the present invention relates to compounds of general formula (1),
wherein
R¹ represents —NR¹⁻²C(O)NR¹⁻³R¹⁻⁴,
wherein R¹⁻² represents hydrogen,
wherein R¹⁻³ represents hydrogen,
wherein R¹⁻⁴ represents C₆- or C₁₀-aryl or pyridyl,
wherein R¹⁻⁴ can optionally be substituted by 1 to 2 substituents C₁-C₄-alkyl, C₁-C₄-alkoxy or halogen,
R² represents hydrogen, halogen, C₁-C₄-alkyl or C₁-C₄-alkoxy, or
R¹ and R² together form a 4-6-membered heterocyclic or heteroaromatic ring, which includes the carbon atoms to which R¹ and R² are bonded and which contains 1 or 2 additional heteroatoms selected from the group oxygen and nitrogen and which contains 1 or 2 double bonds,
   wherein the ring formed by R¹ and R² can optionally be substituted by —NH—C₆— or C₁₀-aryl,
   wherein C₆— or C₁₀-aryl can optionally be substituted by 1 to 2 substituents halogen, C₁-C₄-alkyl or C₁-C₄-alkoxy,
R³ represents hydrogen, C₁-C₁₀-alkyl, —(CH₂)ₘ—C₆- or C₁₀-aryl, —(CH₂)ₘ-C₃-C₇-cycloalkyl, —(CH₂)ₘ-heterocyclyl, —(CH₂)m-heteroaryl,
wherein m represents an integer of one to four,
wherein R³ can optionally be substituted by 1 to 2 radicals R³⁻¹,
wherein R³⁻¹ represents —OR³⁻², —NR³⁻³R³⁻⁴, —C(O)R³⁻², halogen, cyano, nitro, oxo, C₆- or C₁₀-aryl, heterocyclyl, heteroaryl,
wherein R³⁻² represents hydrogen or C₁-C₄-alkyl,
and wherein R³⁻³ and R³⁻⁴ are identical or different and represent hydrogen or C₁-C₄-alkyl,
R⁴ represents hydrogen, halogen, C₁-C₄-alkyl or C₁-C₄-alkoxy,
R⁵ represents hydrogen, C₁-C₁₀-alkyl, —(CH₂)ₙ—C₆— or C₁₀-aryl, —(CH₂)ₙ—C₃-C₇-cycloalkyl, —(CH₂)ₙ-heterocyclyl, —(CH₂)ₙ-heteroaryl,
wherein n represents an integer of one to three,
wherein R⁵ can optionally be substituted by 1 to 2 radicals R⁵⁻¹,
wherein R⁵⁻¹ represents C₁-C₄-alkyl, —OR⁵⁻², —NR⁵⁻³R⁵⁻⁴, —C(O)R⁵⁻², halogen, cyano, nitro, oxo, C₆- or C₁₀-aryl, heterocyclyl, heteroaryl,
wherein R⁵⁻² represents hydrogen or C₁-C₄-alkyl,
and wherein R⁵⁻³ and R⁵⁻⁴ are identical or different and represent hydrogen or C₁-C₄-alkyl,
R⁶ represents hydrogen,
R⁷ represents hydrogen or C₁C₄ alkyl,
or R⁷ and R³ together with the carbon atoms to which they are bonded form a cycloalkyl ring,
X represents oxygen or two hydrogen atoms,
and pharmaceutically acceptable salts thereof.

In another preferred embodiment, the present invention relates to compounds of general formula (I),
wherein
R¹ represents —NR¹⁻²C(O)NR¹⁻³R¹⁻⁴,
wherein R¹⁻² represents hydrogen,
wherein R¹⁻³ represents hydrogen,
wherein R¹⁻⁴ represents C₆-aryl,
wherein R¹⁻⁴ is substituted by 1 to 2 substituents C₁-C₄-alkyl,
R² represents hydrogen, or
R¹ and R² together form a 5-membered heterocyclic or heteroaromatic ring, which includes the carbon atoms to which R¹ and R² are bonded and which contains 1 or 2 additional heteroatoms selected from the group oxygen and nitrogen and which contains 1 or 2 double bonds,
   wherein the ring formed by R¹ and R² can optionally be substituted by —NH—C₆ aryl,
   wherein C₆— or C₁₀-aryl can optionally be substituted by 1 to 2 substituents halogen, C₁-C₄-alkyl or C₁-C₄-alkoxy,
R³ represents hydrogen, C₁-C₁₀-alkyl, —(CH₂)ₘ—C₆-aryl, —(CH₂)ₘ—C₃-C₇-cycloalkyl, —(CH₂)ₘ-heterocyclyl, —(CH₂)ₘ-heteroaryl,
wherein m represents an integer of one or two,
wherein R³ can optionally be substituted by 1 to 2 radicals R³⁻¹,
wherein R³⁻¹ represents —OR³⁻², NR³⁻³R³⁻⁴, —C(O)R³⁻², halogen, oxo, C₆— or C₁₀-aryl, heterocyclyl, heteroaryl,
wherein R³⁻² represents hydrogen or C₁-C₄-alkyl,
and wherein R³⁻³ and R³⁻⁴ are identical or different and represent hydrogen or C₁-C₄-alkyl,
R⁴ represents hydrogen; halogen, C₁-C₄-alkyl or C₁-C₄-alkoxy,
R⁵ represents hydrogen, C₁-C₁₀-alkyl, —(CH₂), —C₆-aryl, —(CH₂), —C₃-C₇-cycloalkyl, —(CH₂)ₙ-heterocyclyl, —(CH₂)ₙ-heteroaryl,
wherein n represents an integer of one to three,
wherein R⁵ can optionally be substituted by 1 to 2 radicals R⁵⁻¹,
wherein R⁵⁻¹ represents C₁-C₄-alkyl, —OR⁵⁻², —NR⁵⁻³R⁵⁻⁴, —C(O)R⁵⁻², halogen, cyano, nitro, oxo, C₆- or C₁₀-aryl, heterocyclyl, heteroaryl,
wherein R⁵⁻² represents hydrogen or C₁-C₄-alkyl,
and wherein R⁵⁻³ and R⁵⁻⁴ are identical or different and represent hydrogen or C₁-C₄-alkyl,
R⁶ represents hydrogen,
R⁷ represents hydrogen,
X represents oxygen,
and pharmaceutically acceptable salts thereof.

In another preferred embodiment, the present invention relates to compounds of general formula (I), wherein R¹ represents a group of the formula

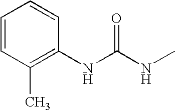

In another preferred embodiment, the present invention relates to compounds of general formula (I), wherein the group of the formula 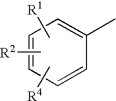
represents a group of the formula

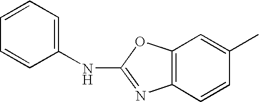

In another preferred embodiment, the present invention relates to compounds of general formula (I), wherein the group of the formula 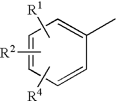
represents a group of the formula

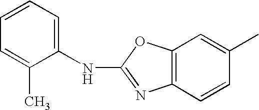

In another more preferred embodiment, the present invention relates to compounds of general formula (I),
wherein R³ represents hydrogen.

In another more preferred embodiment, the present invention relates to compounds of general formula (I),
wherein R⁶ represents hydrogen.

In another more preferred embodiment, the present invention relates to compounds of general formula (I),
wherein R⁷ represents hydrogen.

In a very preferred embodiment, the present invention relates to compounds of general formula (I), wherein the compound is selected from the following group:
4-[(N²-{[4-({[(2-methylphenyl)amino]carbonyl}amino)phenyl]acetyl}-D-lysyl)amino]benzoic acid trifluoroacetate,
4-[(N-[3-(dimethylamino)propyl]-N-{[4-({[(2-methylphenyl)amino]carbonyl}amino)phenyl]acetyl}glycyl)amino]benzoic acid,
4-[(N-(4-aminobutyl)-N-{[4-({[(2-methylphenyl)amino]carbonyl}amino)-phenyl]acetyl}glycyl)amino]benzoic acid,
4-({N-{[4-({[(2-methylphenyl)amino]carbonyl}amino)phenyl]acetyl}-N-[3-(1-pyrrolidinyl)propyl]glycyl}amino)benzoic acid,
4-[(N-[(1-ethyl-2-pyrrolidinyl)methyl]-N-{[4-({[(2-methylphenyl)amino]-carbonyl}amino)phenyl]acetyl}glycyl)amino]benzoic acid,
4-({N-{[4-({[(2-methylphenyl)amino]carbonyl}amino)phenyl]acetyl}-N-[3-(4-phenyl-1-piperazinyl)propyl]glycyl}amino)benzoic acid,
4-{[N-{[4-({[(2-methylphenyl)amino]carbonyl}amino)phenyl]acetyl}-N-(tetrahydro-2-furanylmethyl)glycyl]amino}benzoic acid,
4-{[N-{[4-({[(2-methylphenyl)amino]carbonyl}amino)phenyl]acetyl}-N-(4-piperidinylmethyl)glycyl]amino}benzoic acid,
4-[(N-(3-amino-2,2-dimethylpropyl)-N-{[4-({[(2-methylphenyl)amino]-carbonyl}amino)phenyl]acetyl}glycyl)amino]benzoic acid,
4-({N-{[4-({[(2-methylphenyl)amino]carbonyl}amino)phenyl]acetyl}-N-[2-(1-pyrrolidinyl)ethyl]glycyl}amino)benzoic acid,
4-[(N-{[4-({[(2-methylphenyl)amino]carbonyl}amino)phenyl]acetyl}-N-propylglycyl)amino]benzoic acid,
4-({N-{[4-({[(2-methylphenyl)amino]carbonyl}amino)phenyl]acetyl}-N-[3-(2-oxo-1-pyrrolidinyl)propyl]glycyl}amino)benzoic acid,
4-[(N-(2-methoxyethyl)-N-{[4-({[(2-methylphenyl)amino]carbonyl}amino)-phenyl]acetyl}glycyl)amino]benzoic acid,
4-({N-{[4-({[(2-methylphenyl)amino]carbonyl}amino)phenyl]acetyl}-N-[3-(4-morpholinyl)propyl]glycyl}amino)benzoic acid,
4-{[N-{[4-({[(2-methylphenyl)amino]carbonyl}amino)phenyl]acetyl}-N-(3-pyridinylmethyl)glycyl]amino}benzoic acid,
4-{[N-{[4-({[(2-methylphenyl)amino]carbonyl}amino)phenyl]acetyl}-N-(2-pyridinylmethyl)glycyl]amino}benzoic acid,
4-{[N-{[4-({[(2-methylphenyl)amino]carbonyl}amino)phenyl]acetyl}-N-(4-yridinylmethyl)glycyl]amino}benzoic acid,
4-[(N-[2-(1H-imidazol-4-yl)ethyl]-N-{[4-({[(2-methylphenyl)amino]carbonyl}amino)phenyl]acetyl}glycyl)amino]benzoic acid,
4-({N-{[4-({[(2-methylphenyl)amino]carbonyl}amino)phenyl]acetyl}-N-[2-(2-pyridinyl)ethyl]glycyl}amino)benzoic acid,
4-({N-[(2-anilino-1,3-benzoxazol-6-yl)acetyl]glycyl}amino)benzoic acid,
4-{[N-[(2-anilino-1,3-benzoxazol-6-yl)acetyl]-N-(2-phenylethyl)glycyl]-amino}benzoic acid,
4-({N-[(2-anilino-1,3-benzoxazol-6-yl)acetyl]-N-[2-(2-pyridinyl)ethyl]glycyl}amino)benzoic acid,
4-({N-[(2-anilino-1,3-benzoxazol-6-yl)acetyl]-N-[2-(3,5-dimethoxyphenyl)-ethyl]glycyl}amino)benzoic acid,
4-{[N-({2-[(2-methylphenyl)amino]-1,3-benzoxazol-6-yl}acetyl)glycyl]-amino}benzoic acid,
4-{[N-({2-[(2-methylphenyl)amino]-1,3-benzoxazol-6-yl}acetyl)-N-(2-phenylethyl)glycyl]amino}benzoic acid,
4-({N-({2-[(2-methylphenyl)amino]-1,3-benzoxazol-6-yl}acetyl)-N-[2-(2-pyridinyl)ethyl]glycyl}amino)benzoic acid,
4-[(N-[2-(3-methoxyphenyl)ethyl]-N-{[4-({[(2-methylphenyl)amino]-carbonyl}amino)phenyl]acetyl}glycyl)amino]benzoic acid,
4-[(N-benzyl-N-{[4-({[(2-methylphenyl)amino]carbonyl}amino)phenyl]-acetyl}glycyl)amino]benzoic acid,
4-({N-[(2-anilino-1,3-benzoxazol-6-yl)acetyl]-N-[2-(3-methoxyphenyl)ethyl]-glycyl}amino)benzoic acid,
4-[(N-{[4-({[(2-methylphenyl)amino]carbonyl}amino)phenyl]acetyl}-L-phenylalanyl)amino]benzoic acid,
4-({N-[(2-anilino-1,3-benzoxazol-6-yl)acetyl]-L-phenylalanyl}amino)benzoic acid,
4-[(4-bromo-N-{[4-({[(2-methylphenyl)amino]carbonyl}amino)phenyl]-acetyl}-L-phenylalanyl)amino]benzoic acid
4-[(N-{[4-({[(2-methylphenyl)amino]carbonyl}amino)phenyl]acetyl}glycyl)amino]benzoic acid
4-{[(2S)-4-amino-2-({[4-({[(2-methylphenyl)amino]carbonyl}amino)phenyl]acetyl}amino)butanoyl]amino}benzoic acid
4-[(N²-{[4-({[(2-methylphenyl)amino]carbonyl}amino)phenyl]acetyl}-L-ornithyl)amino]benzoic acid
4-[(N-{[4-({[(2-methylphenyl)amino]carbonyl}amino)phenyl]acetyl}-L-α-aspartyl)amino]benzoic acid
4-[(N-{[4-({[(2-methylphenyl)amino]carbonyl}amino)phenyl]acetyl}-L-tryptophyl)amino]benzoic acid
4-{[N-{[4-(f{[(2-methylphenyl)amino]carbonyl}amino)phenyl]acetyl}-3-(4-pyridinyl)-L-alanyl]amino}benzoic acid
4-{[N-{[4-({[(2-methylphenyl)amino]carbonyl}amino)phenyl]acetyl}-3-(3-pyridinyl)-L-alanyl]amino}benzoic acid
4-{[N-{[4-({[(2-methylphenyl)amino]carbonyl}amino)phenyl]acetyl}-3-(1,3-thiazol-4-yl)-L-alanyl]amino}benzoic acid
4-[(N-{[4-({[(2-methylphenyl)amino]carbonyl}amino)phenyl]acetyl}-L-histidyl)amino]benzoic acid
4-{[(1-{[4-({[(2-methylphenyl)amino]carbonyl}amino)phenyl]acetyl}-2-piperazinyl)carbonyl]amino}benzoic acid
4-[3-({[4-({[(2-methylphenyl)amino]carbonyl}amino)phenyl]acetyl}amino)-1-piperidinyl]benzoic acid
4-[3-({[4-({[(2-methylphenyl)amino]carbonyl}amino)phenyl]acetyl}amino)-1-pyrrolidinyl]benzoic acid
4-[isobutyl(N-{[4-({[(2-methylphenyl)amino]carbonyl}amino)phenyl]acetyl}glycyl)amino]benzoic acid
4-[isobutyl(N-(3-methoxypropyl)-V-{[4-({[(2-methylphenyl)amino]carbonyl}amino)phenyl]acetyl}glycyl)amino]benzoic acid and
4-[(N-(3-methoxypropyl)-N-{[4-({[(2-methylphenyl)amino]carbonyl}amino)phenyl]acetyl}glycyl)(methyl)amino]benzoic acid.

A preferred process for preparation of compounds of general formula (I) has also been found, which comprises reaction of carboxylic acids of general formula (V) 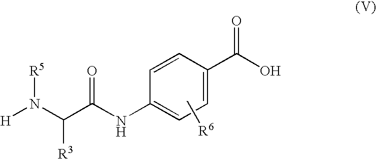
or activated derivatives thereof,
with compounds of the general formula (VI) 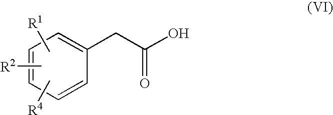
   in the presence of a coupling agent and a base in inert solvents, which will be described in more detail in the descriptive part of the specification.

For the treatment of the above-mentioned diseases, the compounds according to the invention can exhibit non-systemic or systemic activity, wherein the latter is preferred. To obtain systemic activity the active compounds can be administered, among other things, orally or parenterally, wherein oral administration is preferred.

For parenteral administration, forms of administration to the mucous membranes (i.e. buccal, lingual, sublingual, rectal, nasal, pulmonary, conjunctival or intravaginal) or into the interior of the body are particularly suitable. Administration can be carried out by avoiding absorption (i.e. intracardiac, intra-arterial, intravenous, intraspinal or intralumbar administration) or by including absorption (i.e. intracutaneous, subcutaneous, percutaneous, intramuscular or intraperitoneal administration).

For the above purpose the active compounds can be administered per se or in administration forms.

Suitable administration forms for oral administration are, inter alia, normal and enteric-coated tablets, capsules, coated tablets, pills, granules, pellets, powders, solid and liquid aerosols, syrups, emulsions, suspensions and solutions. Suitable administration forms for parenteral administration are injection and infusion solutions.

The active compound can be present in the administration forms in concentrations of from 0.001-100% by weight; preferably the concentration of the active compound should be 0.5-90% by weight, i.e. quantities which are sufficient to allow the specified range of dosage.

The active compounds can be converted in the known manner into the abovementioned administration forms using inert non-toxic pharmaceutically suitable auxiliaries, such as for example excipients, solvents, vehicles, emulsifiers and/or dispersants.

The following auxiliaries can be mentioned as examples: water, solid excipients such as ground natural or synthetic minerals (e.g. talcum or silicates), sugar (e.g. lactose), non-toxic organic solvents such as paraffins, vegetable oils (e.g. sesame oil), alcohols (e.g. ethanol, glycerol), glycols (e.g. polyethylene glycol), emulsifying agents, dispersants (e.g. polyvinylpyrrolidone) and lubricants (e.g. magnesium sulphate).

In the case of oral administration tablets can of course also contain additives such as sodium citrate as well as additives such as starch, gelatin and the like. Flavour enhancers or colorants can also be added to aqueous preparations for oral administration.

For the obtainment of effective results in the case of parenteral administration it has generally proven advantageous to administer quantities of about 0.001 to 100 mg/kg, preferably about 0.01 to 1 mg/kg of body weight. In the case of oral administration the quantity is about 0.01 to 100 mg/kg, preferably about 0.1 to 10 mg/kg of body weight.

It may nevertheless be necessary to use quantities other than those mentioned above, depending on the body weight concerned, the method of administration, the individual response to the active compound, the type of preparation and the time or interval of administration.

Suitable pharmaceutically acceptable salts of the compounds of the present invention that contain an acidic moiety include addition salts formed with organic or inorganic bases. The salt forming ion derived from such bases can be metal ions, e.g., aluminum, alkali metal ions, such as sodium of potassium, alkaline earth metal ions such as calcium or magnesium, or an amine salt ion, of which a number are known for this purpose. Examples include ammonium salts, arylalkylamines such as dibenzylamine and N,N-dibenzylethylenediamine, lower alkylamines such as methylamine, t-butylamine, procaine, lower alkylpiperidines such as N-ethylpiperidine, cycloalkyl-amines such as cyclohexylamine or dicyclohexylamine, 1-adamantylamine, benzathine, or salts derived from amino acids like arginine, lysine or the like. The physiologically acceptable salts such as the sodium or potassium salts and the amino acid salts can be used medicinally as described above and are preferred.

Suitable pharmaceutically acceptable salts of the compounds of the present invention that contain a basic moiety include addition salts formed with organic or inorganic acids. The salt forming ion derived from such acids can be halide ions or ions of natural or unnatural carboxylic or sulfonic acids, of which a number are known for this purpose. Examples include chlorides, acetates, trifluoroacetates, tartrates, or salts derived from amino acids like glycine or the like. The physiologically acceptable salts such as the chloride salts, the trifluoroacetic acid salts and the amino acid salts can be used medicinally as described below and are preferred.

These and other salts which are not necessarily physiologically acceptable are useful in isolating or purifying a product acceptable for the purposes described below.

The salts are produced by reacting the acid form of the invention compound with an equivalent of the base supplying the desired basic ion or the basic form of the invention compound with an equivalent of the acid supplying the desired acid ion in a medium in which the salt precipitates or in aqueous medium and then lyophilizing. The free acid or basic form of the invention compounds can be obtained from the salt by conventional neutralization techniques, e.g., with potassium bisulfate, hydrochloric acid, sodium hydroxide, sodium bicarbonate, etc.

The compounds according to the invention can form non covalent addition compounds such as adducts or inclusion compounds like hydrates or clathrates. This is known to the artisan and such compounds are also object of the present invention.

The compounds according to the invention can exist in different stereoisomeric forms, which relate to each other in an enantiomeric way (image and mirror image) or in a diastereomeric way (image different from mirror image). The invention relates to the enantiomers and the diastereomers as well as their mixtures. They can be separated according to customary methods.

The compounds according to the invention can exist in tautomeric forms. This is known to the artisan and such compounds are also object of the present invention.

General Compound Synthesis

The synthesis of compounds according to the general formula (1) can be illustrated by the following scheme 1:

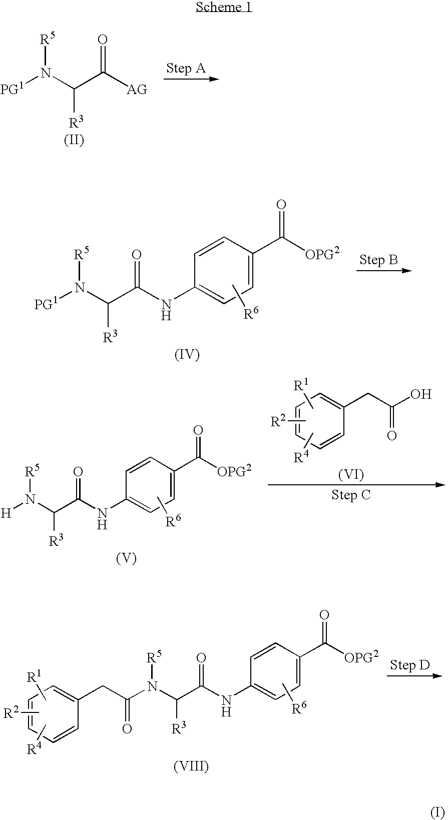

By coupling of the carboxylic acids or activated derivatives (II) with the amines (III), followed by removal of the protecting group PG¹ the amides (V) can be obtained. Coupling with the carboxylic acids (VI) followed by removal of the protecting group PG² affords carboxylic acids of type (VIII.

In the above scheme, AG stands for hydroxyl or a suitable activating group forming an activated carboxylic acid derivative. Activated carboxylic acids derivatives of this type are known to the person skilled in the art and are described in detail in standard textbooks such as, for example in (i) Houben-Weyl, Methoden der organischen Chemie [Methods of Organic Chemistry], Georg mhieme Verlag, Stuttgart or (ii)

Comprehensive Organic Synthesis, Ed. B. M. Trost, Pergamon Press, Oxford, 1991. The carboxylic acid is preferably activated as mixed anhydride, such as, for example, AG=iso-butyl-carbonate; as N-carboxyanhydride (R⁵ and AG=—CO—); or by a coupling agents such as, for example dicyclohexylcarbodiimid (DCC), 1-ethyl-3-(3′-dimethylaminopropyl)carbodiimide×HCl (EDCI), 2-(7-aza-3-oxido-1H-1,2,3-benzo-triazol-1-yl)-1,1,3,3-tetramethyluronium hexafluorophosphate. Other activated carboxylic acid derivatives such as, for example symmetric anhydrides, halides, or activated esters e.g. succinyl or pentafluorophenyl esters may also be employed.

In the above scheme PG¹ stands for a suitable protecting group of the amino group that is stable under the respective reaction conditions. Protecting groups of this type are known to the person skilled in the art and are described in detail in T. W. Greene, P. G. Wuts, Protective Groups in Organic Synthesis, 3^{rd} ed., John Wiley, New York, 1999. The amino group is preferably protected by carbamates, PG¹ being for example tert-butyloxycarbonyl (Boc), 9-fluorenylmethyloxycarbonyl (FMOC) or benzyloxy-carbonyl (Cbz-/Z-) or other oxycarbonyl derivatives.

In the above scheme PG² stands for a suitable protecting group of the carboxyl group or COOPG² stands for the carboxylic group attached to a polymeric resin suitable for solid phase synthesis. Protecting groups of this type are known to the person skilled in the art and are described in detail in T. W. Greene, P. G. Wuts, Protective Groups in Organic Synthesis, 3^{rd} ed., John Wiley, New York, 1999. The carboxyl group is preferably esterified, PG² being C₁₋₆-alkyl such as, for example, methyl, ethyl, propyl, isopropyl, butyl, isobutyl, t-butyl, pentyl, isopentyl, neopentyl, hexyl, a C₃₋₇-cycloalkyl such as, for example, cyclopropyl, cyclopropylmethyl, cyclobutyl, cyclo-pentyl, cyclohexyl, an aryl such as, for example, phenyl, benzyl, tolyl or a substituted derivative thereof.

Step A

Formation of the amides (IV) can take place by reacting an activated form of the respective carboxylic acid (II), such as a N-carboxyanhydride or an iso-butylcarbonate with the desired amine (III) or an acceptable salt thereof

N-carboxyanhydrides of (II) are commercially available or can be prepared for example by the reaction of the Bis-(N-tert-butyloxycarbonyl) protected derivative of (II) with thionylchloride and pyridine in dimethylformamide or by the reaction of the free amino acid of (II) with phosgene or with phosgene equivalents such as diphosgene, triphosgene or methylchloroformate. Iso-butylcarbonates can be prepared in situ by reaction of the N-protected amino acid (II) with iso-butylchloroformate as described below. Activated derivatives of the acids (II) such as other anhydrides, halides, esters e.g. succinyl or pentafluorophenyl esters or activated carboxylic acids obtained by the reaction with coupling agents such as, for example dicyclohexyl-carbodiimid (DCC), 1-ethyl-3-(3′-dimethylaminopropyl)carbodiimide×HCl (EDCI), 2-(7-aza-3-oxido-1H-1,2,3-benzotriazol-1-yl)-1,1,3,3-tetramethyluronium hexafluorophosphate may also be employed.

For example, amides of type (IV) can be prepared as follows:

1) N-carboxyanhydride Procedure

A solution/suspension of the amine (III), the N-carboxyanhydride of (II) and catalytic amounts of 4-(N,N′-dimethylamino)pyridine in an inert solvent was refluxed for 0.5-14 days with exclusion of moisture. The product was either isolated by filtration or by aqueous workup employing standard procedures. If necessary the product was purified by trituration or by flash-chromatography or used without further purification.

2) Mixed Anhydride Procedure

A solution of the carboxylic acid derivative (II) and of N-methylmorpholine in an inert solvent was cooled to −15° C. and iso-butyl chloroformate was added and stirred at 0° C. The amine (III) in an inert solvent was added at −15° C. The solution was stirred at 0° C., and at r.t. and was evaporated. The residue was redissolved in ethyl acetate, washed with aqueous acid and base, dried and evaporated. If necessary the product was purified by trituration or by flash-chromatography or used without further purification.

Compounds of general formula (II) are commercially available, known or can be prepared by customary methods starting from known α-amino acids or precursors for customary α-amino acid synthesis. For the preparation process according to the invention, the amino group is in this case blocked by a conventional protective group PG¹.

In the α-position to the carboxyl group, these carboxylic acid derivatives can have substituents such as described under R³ and R⁴, for example, hydrogen, a C₁-C₁₀-alkyl, a C₃-C₇-cycloalkyl, an aryl, an alkenyl residue, or an alkinyl residue. The alkyl, alkenyl and cycloalkyl residues and the benzyl residue can be introduced by reaction of the ester of the starting compounds with the appropriate alkyl, alkenyl, cycloalkyl or benzyl halides in basic medium, if the corresponding derivatives are not commercially available. The alkinyl residue can be introduced, for example, by reaction of the bromo ester of the present starting compound with an appropriate acetylide anion. In the case of the phenyl residue the starting materials used are preferably the corresponding α-phenyl-α-aminocarboxylic acid derivatives and, if necessary, the other substituents at the α-C atom to the terminal carboxyl group are introduced via the appropriate alkyl halide.

The above reactions and their implementation are well known to the person skilled in the art and are described in detail in standard textbooks such as, for example, in (i) Houben-Weyl, Methoden der organischen Chemie [Methods of Organic Chemistry], Georg Thieme Verlag, Stuttgart or Stuttgart or (ii) Comprehensive Organic Synthesis, Ed. B. M. Trost, Pergamon Press, Oxford, 1991.

If the substituents themselves should be substituted, e.g. by R′, appropriate reactive groups should be present in the substituent to allow further functionalization. These reactive groups should be inert to the reaction conditions of the previous step. For this purpose, the substituent can also be unsaturated to allow further functionalization such as palladium catalyzed C—C-coupling reactions (e.g. Heck-reaction or Sonoga-shira-reaction), eventually followed by hydrogenation (scheme 2):

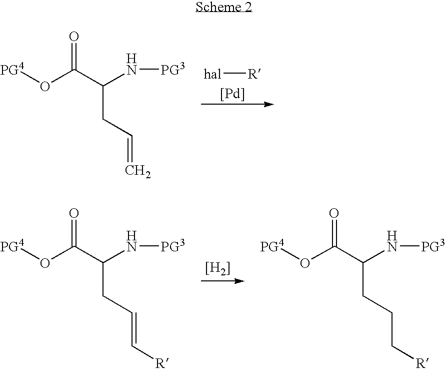

In the abovementioned scheme PG⁴ stands for a protecting group of the carboxyl group as described under PG², hal stands for a leaving group such as a halogen, tosyl, mesyl or triflate, [Pd] stands for a Palladium(0) or Palladium(II) moiety. PG³ stands for a protecting group of the amino group such as described under PG¹. Protecting groups of this type are known to the person skilled in the art and are described in detail in T. W. Greene, P. G. Wuts, Protective Groups in Organic Synthesis, 3^{rd} ed., John Wiley, New York, 1999.

If the substituent R³ in the α-position to the carboxylic group carry an appropriate substituted aryl or heteroaryl unit, another method for insertion of an additional substituent are the C—C-coupling reactions as described under the synthesis of precursors (VI).

Compounds of general formula (III) are commercially available, known or can be prepared by customary methods starting from known carboxylic acid derivatives.

Step B

The removal of protecting group PG¹ can be performed, depending on the nature of PG¹, either by an acid such as trifluoroacetic acid (for example in the case PG¹ is tert-butyloxycarbonyl (Boc)), a base such as piperidine (for example in the case PG¹ is 9-fluorenylmethyloxycarbonyl (FMOC)) or by catalytic hydrogenation (for example in the case PG¹ is benzyloxycarbonyl (Cbz-/Z-)).

Step C

Formation of the amides (VII) can take place by reacting the respective carboxylic acids (VI)— activated by a coupling agent such as DCC and HOBt; EDCI and HOBt or HATU—with the desired amines (V) or an acceptable salt thereof. Activated derivatives of the acids (VI) such as anhydrides, halides, and esters e.g. succinyl or pentafluorophenyl esters may also be employed.

For example, amides (VII) can be prepared as follows:

A solution of carboxylic acid, HOBt and EDCI in an inert solvent is stirred at r.t. After addition of the amine and a non-nucleophilic base such as ethylisopropylamine stirring is continued at r.t. or elevated temperature. The reaction mixture is poured into water and worked up by standard procedures.

Compounds of general formula (VI) are commercially available, known or can be prepared by customary methods starting from known carboxylic acid derivatives.

For example, biphenyl substituted acetic acid derivatives can be prepared by means of an aryl-aryl coupling of the respective phenyl acetic acid derivatives and a suitable phenyl system.

Possible coupling reactions are, for example, the reaction of two unsubstituted phenyl groups in the presence of AlCl₃ and an acid (Scholl reaction), the coupling of the two phenyl iodides in the presence of copper (Ullmann reaction), the reaction of the unsubstituted carboxylic acid derivative with a phenyldiazonium compound under basic conditions (Gomberg-Bachmann reaction) or coupling with participation of organometallic reagents such as coupling of a phenyl halide with an organometallic phenyl compound in the presence of a palladium compound, for example, a Pd(0), a Pd(II) or a Pd(IV) compound, and of a phosphane such as triphenylphosphane (e.g. Suzuki reaction).

Bisarylureas can be prepared by coupling of an amino phenyl acetic acid derivative and a phenylisocyanate. Bisarylamides can be prepared by coupling of an amino phenyl acetic acid and an activated benzoic acid derivative such as described under Step A. Bisarylcarbamates can be prepared by coupling of an isocyanato phenyl acetic acid ester and a phenol derivative followed by saponification as described in Step D.

Anilinobenzoxazoles can be prepared by coupling of arylisothiocyanates with ortho-amino-hydroxyphenyl derivatives and subsequent cyclization to the corresponding anilino-benzoxazole derivatives in the presence of suitable desulfurization reagents, for example carbodiimides or mercury(II) salts.

Step D

The removal of the protecting group PG² can be performed either by an acid such as trifluoroacetic acid or an base such as potassium hydroxide or lithium hydroxide, depending on the nature of PG². Reactions are carried out in aqueous, inert organic solvents such as alcohols e.g. methanol or ethanol, ethers e.g. tetrahydrofurane or dioxane or polar aprotic solvents e.g. dimethylformamide. If necessary, mixtures of the above solvents may be used.

In case PG² stands for polymeric resin, the removal can take place using strong acid such as trifluoroacetic acid in dichloromethane.

### EXAMPLES

Abbreviations

| | |
|---|---|
| AcOH | acetic acid |
| Boc | tert-butyloxycarbonyl |
| DCC | dicyclohexylcarbodiimid |
| GC | gas chromatography |
| DIPEA | diisopropylethylamine |
| DMF | dimethylformamide |
| EDCI | 1-ethyl-3-(3'-dimethylaminopropyl)carbodiimide × HCl |
| eq. | equivalents |
| FC | flash chromatography |
| HATU | 2-(7-aza-3-oxido-1H-1,2,3-benzotriazol-1-yl)- |
| | 1,1,3,3-tetramethyluronium hexafluorophosphate |
| HOBt | N-hydroxybenzotriazole monohydrate |
| HPLC | high performance liquid chromatography |
| ICAM-1 | intracellular adhesion molecule 1 |
| IL-1 | interleukin 1 |
| LPS | lipopolysaccharide |
| MAdCAM-1 | mucosal addressin cell adhesion molecule 1 |
| MeOH | methanol |
| MeCN | acetonitrile |
| min. | minutes |
| M.p. | melting point |
| NF-κB | nuclear factor κB |
| NMR | nuclear magnetic resonance |
| n.d. | not determined |
| r.t. | room temperature |
| R_{f} | TLC: R_{f} value = distance spot traveled/ |
| | distance solvent front traveled |
| TFA | trifluoroacetic acid |
| THF | tetrahydrofurane |
| TLC | thin layer chromatography |
| TNF-α | tumor necrosis factor α |
| t_{R} | retention time determined by HPLC |
| VCAM-1 | vascular cell adhesion molecule 1 |
| VLA-4 | very late antigen 4 (α₄β₁ integrin) |

General remarks

In the examples below, all quantitative data, if not stated otherwise, relate to percentages by weight.

For synthetic process some compounds are immobilized on solid phase. A preferred polymeric resin for this purpose is Wang polystyrene resin (Rapp-Polymere, Tüibingen). As known to the one skilled in the art, the compounds can also be prepared by liquid synthetic methods using essentially the same reagents. In this case Wang polystyrene resin is substituted by an protection group for carboxyl groups such as esters.

Flash chromatography was carried out on silica gel 60, 40-63 μm (E. Merck, Darmstadt, Germany).

Thin layer chromatography was carried out, employing silica gel 60 F₂₅₄ coated aluminum sheets (E. Merck, Darmstadt, Germany) with the mobile phase indicated.

Melting points were determined in open capillaries and are not corrected.

All retention times are indicated in minutes and, if not stated otherwise, were determined by high-performance liquid chromatography (HPLC) by means of UV detection at 210 or 214/250 μm, at a flow rate of 1 ml/min at ambient temperature with linear gradients. An MeCN/H₂O mixture with 0.1% TFA (vol./vol.) was used as eluent.

Method A:

Column: LiChrospher 100 RP-18, 5 μm, 250×4 mm (E. Merck, Darmstadt, Germany)

Gradient: 0 min MeCN/H₂O 0:100, 25 min MeCN/H₂O 100:0, 31 min MeCN/H₂O 100:0, 32 min MeCN/H₂O 0:100, 38 min MeCN/H₂O 0:100.

Method B:

Column: Purospher RP-18, 5 μm, 250×4 mm (E. Merck, Darmstadt, Germany).

Gradient: 0 min MeCN/H₂O 0:100, 25 rmin MeCN/H₂O 100:0, 31 min MeCN/H₂O 100:0, 32 min MeCN/H₂O 0:100, 38 min MeCN/H₂O 0:100.

Method C:

Column: Eurospher 100, C18, 5 μm, 120×4 mm (Knauer, Berlin, Deutschland).

Gradient: 0 min MeCN/H₂O 10:90, 13 min MeCN/H₂O 80:20, 15 min MeCN/H₂O 80:20, 17 min MeCN/H₂O10:90.

Method D

Column: LiChrospher 100 RP-18, 5 μm, 250×4 mm (E. Merck, Darmstadt, Germany).

Gradient: 0 min MeCN/H₂O 10:90, 25 min MeCN/H₂O 100:0, 31 min MeCN/H₂O 100:0, 32 min MeCN/H₂O 10:90, 38 min MeCN/H₂O 10:90.

The mass determinations were carried out using the electron spray ionization (ESI) method employing loop injection or split injection via a HPLC system.

Precursor Synthesis

### Example I

### 2-{4-[(2-Toluidinocarbonyl)amino]phenyl}acetic acid

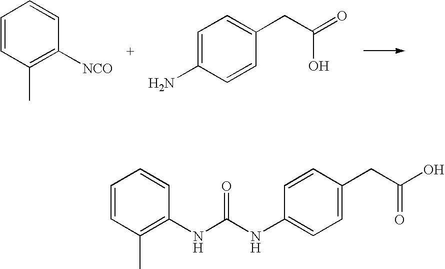

To a solution of 2-(4-aminophenyl)acetic acid (108.8 g, 0.72 mol) in CH₂Cl₂ (1.0 l) and triethylamine (120 ml) was added a solution of 2-methylphenyl isocyanate (90.5 ml, 0.72 mol) in CH₂Cl₂ (500 ml) dropwise at r.t. After stirring for 18 h at r.t., water (2.5 l) and CH₂Cl₂ (2.0 l) were added and the layers were separated. The organic layer was extracted with water (3×400 ml). The combined aqueous layers were concentrated to 3.0 l and acidified to pH 2 by the addition of concentrated aqueous HCl. The precipitate was collected by filtration, washed with cold water and dried in an exsiccator over concentrated H₂SO₄ affording 166.5 g (82%) white solid. M.p. 205-206° C.; TLC (CH₂Cl₂/MeOH 9:1): R_{f} 0.14. ¹H-NMR (400 MHz, D₆-DMSO): 12.21 (br s, 1H), 9.11 (s, 1H), 8.00 (s, 1H), 7.83 (d, 7.6 Hz, 1H), 7.40 (d, 8.5 Hz, 2H), 7.17-7.12 (m, 4H), 6.96-6.92 (m, 1H), 3.48 (s, 2H), 2.24 (s, 3H).

### Example IV

### Methyl 4-({[(3-methoxypropyl)amino]acetyl}amino)benzoate

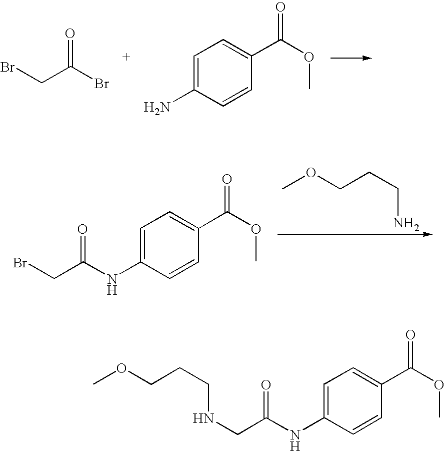

To a solution of methyl 4-aminobenzoate (10.0 g, 66.2 mmol) and triethylamine (10.1 ml, 72.8 mmol) in dichloromethane (100 ml) was added a solution of bromo-acetylbromide (6.34 ml, 72.8 mmol) in dichloromethane (30 ml) at 0° C. After stirring for 18 h at room temperature and 18 h under reflux the reaction mixture was concentrated under vacuum. The residue was taken up in ethyl acetate, washed with 1 N aqueous HCl and water, dried over MgSO₄ and evaporated. Yield 15.8 g (88%) of methyl 4-[(bromoacetyl)amino]benzoate as a pale brown solid. M.p.: 144-146° C., TLC (hexane/ethyl acetate 1:1): R_{f} 0.46; ¹H-NMR (400 MHz, D₆-DMSO) 10.77 (s, 1H), 7.95 (d, 8.7 Hz, 2), 7.73 (d, 8.7 Hz, 2H), 4.08 (s, 2H), 3.83 (s, 3H); ESI-MS: [M+H⁺]=271.8

To a solution of methyl 4-[(bromoacetyl)amino]benzoate (2.72 g, 10.0 mmol) in dimethylformamide (20 ml) was added 3-methoxypropylamine (1.78 g, 20.0 mmol) and triethylamine (22.3 ml, 160 mmol). After stirring at room temperature for 18 h, the reaction mixture was concentrated under vacuum and purified by flash chromatography (CH₂Cl₂/MeOH 9:0.4) affording 1.81 g (65%) of methyl 4-({[(3-methoxy-propyl)amino]acetyl}amino)benzoate as a pale red solid. M.p.: 49-50° C., TLC (CH₂Cl₂/MeOH 9:1): R_{f} 0.36; ¹H-NMR (400 MHz, D₆-DMSO): 10.10 (br s, 1H), 7.92 (d, 8.7 Hz, 2H), 7.77 (d, 8.7 Hz, 2H), 3.82 (s, 3H), 3.40-3.30 (m, 4H), 3.21 (s, 3H), 2.57 (t, 7.0 Hz, 2H), 1.69-1.63 (m, 2H) (amine-H not observed); ESI-MS: [M+H⁺]=281.0

### Example VIII

### Ethyl 4-{3-[(tert-butoxycarbonyl)amino]-1-piperidinyl}benzoate

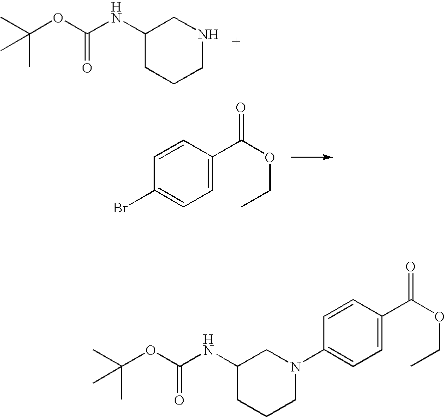

To a solution of ethyl 4-bromobenzoate (1.10 g, 4.80 mmol) in toluene (15 ml) was added tert-butyl 3-piperidinylcarbamate (1.16 g, 5.80 mmol), cesium carbonate (2.20 g, 6.70 mmol), palladium(II)acetate (35 mg, 156 μmol) and (+/−)-2,2′-bis-(diphenylphosphino)-1,1′-binaphthaline (BINAP) (140 mg, 225 μmol). After stirring for 3 days at 100° C., the reaction mixture was diluted with tert-butylmethylether (25 ml) and was filtered. The filtrate was washed with brine, dried over MgSO₄, concentrated and purified by flash chromatography (CH₂Cl₂/MeOH 99:0.6 then 99:1) affording 849 mg (51%) pale yellow solid. M.p.: 107-108° C., TLC (CH₂Cl₂/MeOH 9:0.2): R_{f} 0.66; ¹H-NMR (400 MHz, D₆-DMSO) 7.76 (d, J=8.9 Hz, 2H), 6.96-6.92 (m, 3H), 4.23 (q, J=7.1 Hz, 2H), 3.81-3.73 (m, 2H), 3.41-3.35 (m, 1H), 2.88-2.72 (m, 2H), 1.84-1.70 (m, 2H), 1.50-1.40 (m, 2H), 1.40 (s, 9H), 1.28 (t, J=7.1 Hz, 3H); EI-MS: [M⁺]=348.

### Example IX

### Ethyl 4-(isobutylamino)benzoate

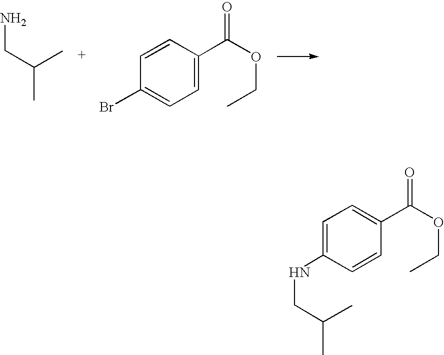

To a solution of ethyl 4-bromobenzoate (40.0 g, 175 mmol) in toluene (280 ml) was added iso-butylamine (15.3 g, 210 mmol), cesium carbonate (79.7 g, 244 mmol), palladium(II)acetate (290 mg, 1.31 mmol) and (+/−)-2,2′-bis-(diphenylphosphino)-1,1′-binaphthaline (BINAP) (1.30 g, 1.96 mmol). After stirring for 1 day at 100° C., the same amounts of palladium(II)acetate and BINAP were added and stirring at 100° C. was continued for 4 days. The reaction mixture was diluted with tert-butylmethylether (100 ml) and was filtered. The filtrate was washed with brine, dried over MgSO₄, concentrated and purified by flash chromatography (CH₂Cl₂/MeOH 9:0.3 then 9:0.5) affording 25.0 g (65%) white solid. M.p.: 57-58° C., TLC (CH₂Cl₂): R_{f} 0.44, ¹H-NMR (400 MHz, D₆-DMSO) 7.67 (d, J=8.8 Hz, 2H), 6.59-6.55 (m, 3H), 4.20 (q, J=7.1 Hz, 2H), 2.88 (t, J=6.0 Hz, 2H), 1.88-1.78 (m, 1H), 1.26 (t, J=7.1 Hz, 3H), 0.93, (d, J=6.7 Hz, 6H); ESI-MS: 222.2 [M+H]⁺.

### Example X

### (2-anilino-1,3-benzoxazol-6-yl)acetic acid

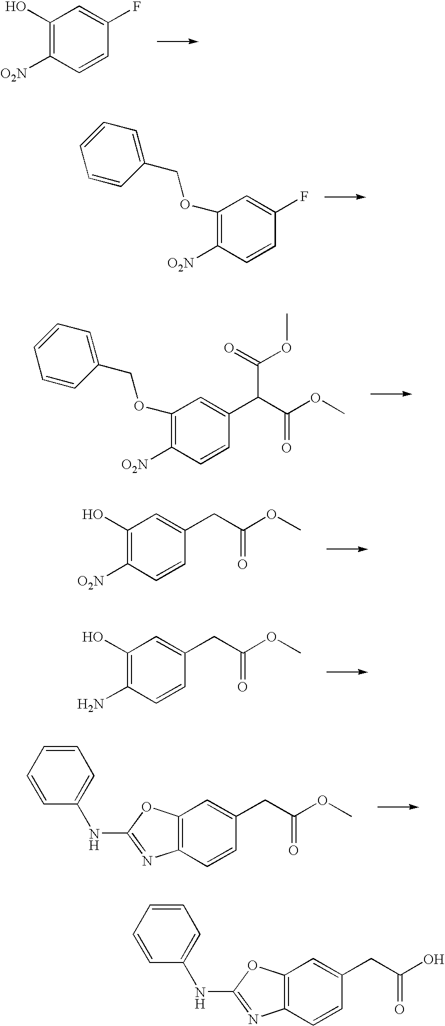

To a stirred solution of 2-nitro-5-fluorophenole (44.4 g, 283 mmol) in acetonitrile (357 ml) was added potassium carbonate (39.1 g, 283 mmol). After dropwise addition of benzylbromide (50.8 g, 297 mmol), the reaction mixture was refluxed for 2 h. Water (1 l) was added and the resulting solution was extracted with tert-butylmethylether (4×). The combined organic layers were washed with brine, dried over MgSO₄ and evaporated, affording 68.9 g (99%) of 2-(benzyloxy)-4-fluoro-1-nitrobenzene as a yellow solid: M.p. 64-65° C.; TLC (cyclohexane/ethyl acetate 8:2): R_{f} 0.50; ¹H-NMR (400 MHz, D₆-DMSO) 8.05 (dd, 9.1, 6.1 Hz, 1H), 7.49-7.36 (m, 6H), 7.03-6.98 (m, 1H), 5.34 (s, 2H).

To a stirred solution of dimethyl malonate (123 g, 929 mmol) in 1-methyl-2-pyrrolidon (554 ml) was added 60% sodium hydride in mineral oil (40.8 g, 1.02 mol) at room temperature. Stirring was continued until no further gas was formed, and 2-(benzyloxy)-4-fluoro-1-nitrobenzene (140 g, 566 mmol) was added portionwise at room temperature. After stirring at 80° C. for 4 h, the reaction mixture was poured into ice water. The pH was adjusted to 7 by the addition of 5 M HCl, and the reaction mixture was extracted with ethyl acetate. The combined organic layers were washed with water and brine, dried over MgSO₄ and evaporated. The residue was triturated with tert-butylmethylether affording 197 g (97%) of dimethyl 2-[3-(benzyloxy)-4-nitrophenyl]malonate as a pale red viscous oil: TLC (cyclohexane/ethyl acetate 7:3): R_{f} 0.22; ¹H-NMR (400 MHz, D₆-DMSO) 7.92 (d, 8.6 Hz, 1H), 7.51-7.35 (m, 6H), 7.15 (dd, 8.1, 1.5 Hz, 1H), 5.28 (s, 2H), 5.22 (s, 1H), 3.70 (s, 6H); ESI-MS: [M−H]⁻=358.1.

Dimethyl 2-[3-(benzyloxy)-4-nitrophenyl]malonate (30.0 g, 83.5 mmol) was dissolved in a mixture of acetic acid (200 ml) and concentrated hydrochloric acid (100 ml). Heating the reaction mixture to 100° C. resulted in an exothermic gas evolution, which ceased after 2.5 h. Evaporation of the solvent and drying in vacuum afforded 15.4 g (94%) of (3-hydroxy-4-nitrophenyl)acetic acid as a yellow viscous solid, which was employed in the next reaction step without further purification: TLC (cyclo-hexane/ethyl acetate/acetic acid 6:4:0.1): R_{f} 0.14, ¹H-NMR (400 MHz, D₆-DMSO) 12.58 (br s, 1H), 10.98 (s, 1H), 7.85 (d, 8.5 Hz, 1H), 7.05 (d, 1.7 Hz, 1H), 6.88 (dd, 8.5, 1.7 Hz, 1H), 3.64 (s, 2H).

A solution of (3-hydroxy-4-nitrophenyl)acetic acid (14.0 g, 71.0 mmol) and concentrated sulfuric acid (6 ml) in methanol (300 ml) was refluxed for 2 h. After addition of water (1 l), the solution was extracted with tert-butylmethylether (4×). The combined organic layers were washed with saturated NaHCO₃ and brine, dried over MgSO₄ and evaporated. The residue was triturated with petrol ether affording 11.0 g (73%) of methyl (3-hydroxy-4-nitrophenyl)acetate as a yellow solid: M.p. 56-57° C.; TLC (dichloromethane): R_{f} 0.46; ¹H-NMR (400 MHz, D₆-DMSO) 10.98 (s, 1H), 7.86 (d, 8.5 Hz, 1H), 7.05 (d, 1.7 Hz, 1H), 6.88 (dd, 8.5, 1.7 Hz, 1H), 3.76 (s, 2H), 3.63 (s, 3H); GC-MS (EI): [M]⁺=211.

Methyl (3-hydroxy-4-nitrophenyl)acetate (9.5 g, 45.0 mmol) was dissolved in ethanol (150 ml). After addition of 10% Pd—C (0.95 g), the reaction mixture was hydrogenated at atmospheric pressure at room temperature. The catalyst was removed by filtration over celite. Concentrating the filtrate to dryness afforded 7.92 g (97%) methyl (4-amino-3-hydroxyphenyl)acetate as a brown solid: M.p. 112-114° C.; TLC (dichloromethane): R_{f} 0.12; ¹H-NMR (400 MHz, D₆-DMSO) 8.98 (br s, 1H), 6.56 (d, 1.8 Hz, 1H), 6.50 (d, 7.8 Hz, 1H), 6.42 (dd, 7.8, 1.8 Hz, 1H), 4.45 (br s, 2H), 3.57 (s, 3H), 3.39 (s, 2H); GC-MS (EI): [M]+=181.

To a solution of methyl (4-amino-3-hydroxyphenyl)acetate (1.00 g, 5.52 mmol) in ethanol (120 ml) was added dropwise phenylisothiocyanate (0.82 g, 6.07 mmol) at room temperature. After stirring for 2.5 h, 1,3-dicyclohexylcarbodiimide (1.71 g, 8.28 mmol) was added and the reaction mixture was heated to reflux for 3 h. The precipitate was removed by filtration. The filtrate was concentrated to dryness and the residue was taken up in toluene. Undissolved material was removed by filtration and the filtrate was concentrated to dryness. Purification by flash chromatography (cyclohexane/ethyl acetate 9:0.7->6:4) yielded 0.85 g (55%) of methyl (2-anilino-1,3-benzoxazol-6-yl)acetate as a white solid: M.p. 155-157° C.; TLC (cyclohexane/-ethyl acetate 6:4): R_{f} 0.38; ¹H-NMR (400 MHz, D₆-DMSO) 10.68 (s, 1H), 7.76 (d, 7.7 Hz, 2H), 7.42-7.36 (m, 4H), 7.12 (dd, 8.0, 1.4 Hz, 1H), 7.05-7.02 (m, 1H), 3.76 (s, 2H), 3.63 (s, 3H); ESI-MS: [M+H]⁺=282.9.

A solution of methyl (2-anilino-1,3-benzoxazol-6-yl)acetate (3.00 g, 10.6 mmol) and potassium hydroxide (892 mg, 15.9 mmol) in methanol/dioxane/water (200 ml/80 ml/200 ml) was stirred at room temperature for 4 h. The reaction mixture was diluted with water and washed with tert-butylmethylether. The pH was adjusted to 3 by the addition of 1 N hydrochloric acid. The precipitate was collected by filtration and dried in vacuum affording 2.71 g (95%) of (2-anilino-1,3-benzoxazol-6-yl)acetic acid as a white solid: M.p. 222-223° C.; TLC (dichloromethane/methanol/acetic acid 9:0.5:0.1): R_{f} 0.44;. ¹H-NMR (400 MHz, D₆-DMSO): 12.20 (br s, 1H), 10.62 (s, 1H), 7.75 (d, 7.8 Hz, 2H), 7.40-7.35 (m, 4H), 7.11 (d, 8.1 Hz, 1H), 7.05-7.01 (m, 1H), 3.65 (s, 2H); ESI-MS: [M+H]⁺⁼268.9.

### Example XI

### {2-[(2-methylphenyl)amino]-1,3-benzoxazol-6-yl}acetic acid

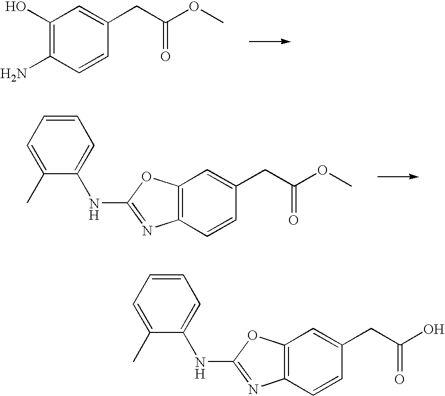

To a solution of methyl (4-amino-3-hydroxyphenyl)acetate (3.50 g, 19.3 mmol) in ethanol (300 ml) was added dropwise o-tolylisothiocyanate (3.17 g, 21.3 mmol) at room temperature. After stirring for 18 h, 1,3-dicyclohexylcarbodiimide (5.98 g, 29.0 mmol) was added and the reaction mixture was heated to reflux for 3 h. The precipitate was removed by filtration. The filtrate was concentrated to dryness and the residue was taken up in toluene. Undissolved material was removed by filtration and the filtrate was concentrated to dryness. Purification by flash chromatography (cyclohexane/ethyl acetate 9:1->6:4) yielded 3.64 g (64%) of methyl {2-[(2-methylphenyl)amino]-1,3-benzoxazol-6-yl}acetate as a white solid. M.p.: 128-129° C.; TLC (cyclohexane/ethyl acetate 6:4): R_{f} 0.38; ¹H-NMR (400 MHz, D₆-DMSO) 9.68 (s, 1H), 7.82 (d, 8.6 Hz, 1H), 7.38 (d, 1.0 Hz, 1H), 7.30-7.23 (m, 3H), 7.10-7.07 (m, 2H), 3.74 (s, 2H), 3.62 (s, 3H), 2.30 (s, 3H); ESI-MS: [M+H]⁺⁼296.9

A solution of methyl {2-[(2-methylphenyl)amino]-1,3-benzoxazol-6-yl}acetate (3.19 g, 10.8 mmol) and potassium hydroxide (909 mg, 16.2 mmol) in methanol/-dioxane/water (70 ml/70 ml/30 ml) was stirred at room temperature for 5 h. The reaction mixture was diluted with water and washed with tert-butylmethylether. The pH was adjusted to 3 by the addition of 1 N hydrochloric acid. The precipitate was collected by filtration and dried in vacuum affording 2.76 g (91%) of {2-[(2-methylphenyl)amino]-1,3-benzoxazol-6-yl}acetic acid as a white solid. M.p.: 199-200° C.; TLC (cyclohexane/ethyl acetate 6:4): R_{f} 0.08;. ¹H-NMR (400 MHz, D₆-DMSO) 9.65 (s, 1H), 7.82 (d, 9.1 Hz, 1H), 7.35 (s, 1H), 7.29-7.24 (m, 3H), 7.10-7.06 (m, 2H), 3.62 (s, 2H), 2.30 (s, 3H); ESI-MS: [+H]⁺=282.9.
Compound Synthesis (In Solution) 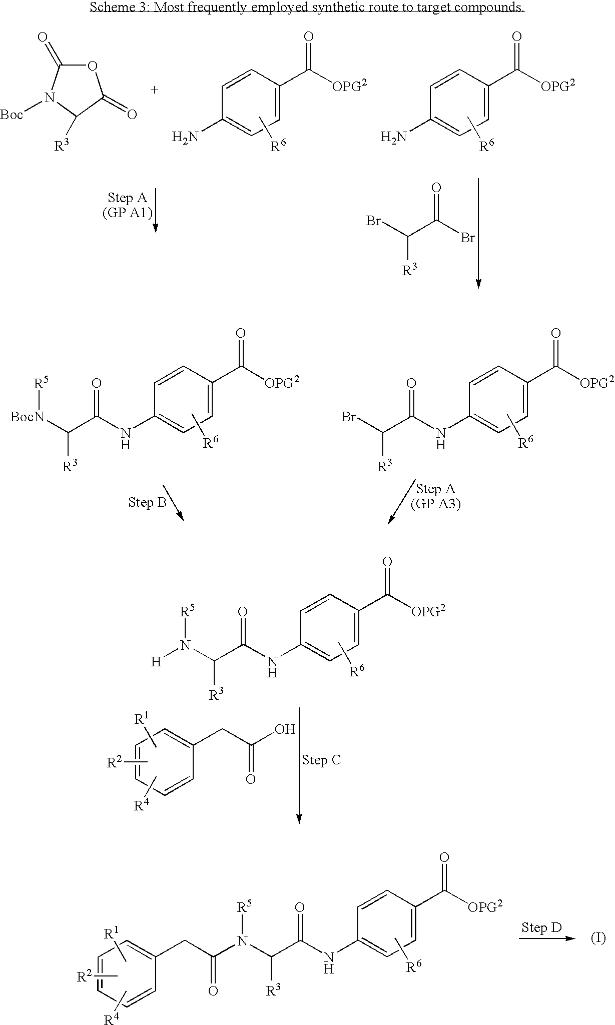
Step A
General Procedure A1 (GP A1): Coupling of amines with Boc-L-leucin-N-carboxy-anhydride:

A solution/suspension of 1.0 eq. of the amine, 1.0 eq. of Boc-L-leucin-N-carboxyanhydride and 0.3 eq. of 4-(N,N′-dimethylamino)pyridine was refluxed for 0.5-14 days with exclusion of moisture. If a precipitate was formed, the precipitate (product) was collected by filtration. The reaction mixture/filtrate was evaporated to dryness, redissolved in ethyl acetate and washed with 1 N aqueous HCl, saturated aqueous NaHCO₃ and brine, dried over MgSO₄ and evaporated. Both solids were combined. If necessary the product was purified by trituration or by flash-chromatography or used without further purification.

### Example 1

### Methyl 4-({Boc-L-leucine}amino)benzoate

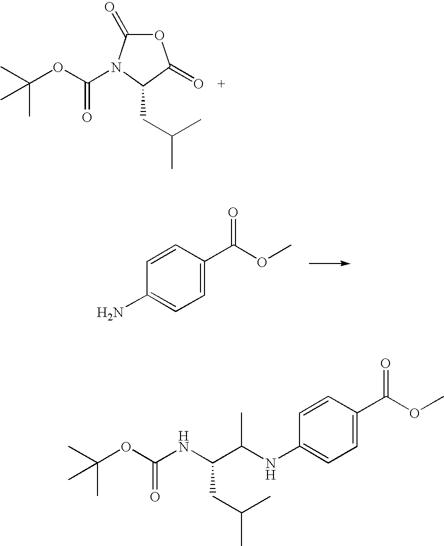

Methyl 4-aminobenzoate (0.75 g, 4.97 mmol) was dissolved in CH₂Cl₂ (7 ml). After the addition of Boc-L-leucin-N-carboxyanhydride (1.28 g, 4.79 mmol) and 4-(N,N′-dimethylamino)pyridine (180 mg, 1.49 mmol) the solution was stirred under reflux for 4 days. The precipitate (product) was collected by filtration. The filtrate was evaporated to dryness, redissolved in ethyl acetate and washed with 1 N aqueous HCl, saturated aqueous NaHCO₃ and brine, dried over MgSO4 and evaporated. Combined Yield: 1.35 (75%) white solid. M.p.: 72-73° C.; TLC (CH₂Cl₂/MeOH 9:0.1): R_{f} 0.52;. ¹H-NMR (400 MHz, D₆-DMSO) 10.32 (s, 1H), 7.93 (d, J=8.7 Hz, 2H), 7.76 (d, J=8.7 Hz, 2H), 7.12 (d, J=7.7 Hz, 1H), 4.18-4.12 (m, 1H), 3.83 (s, 3H), 1.69-1.36 (m, 3H), 1.38 (s, 9H), 0.91-0.88 (m, 6H); ESI-MS: [M+H]⁺=309.1.

General Procedure A2 (GP A2): Coupling of Amines with Carboxylic Acids Activated by Iso-Butyl Chloroformate.

A solution of 1.0 eq. of the carboxylic acid derivative and 1.0 eq. of N-methyl-morpholine in tetrahydrofurane was cooled to −15° C. and 1.0 eq. of iso-butyl chloroformate was added dropwise. After 5 min at 0° C., 1.0 eq. of the amine in tetrahydrofurane was added at −15° C. The solution was stirred for 1 h at 0° C., 1-4 d at r.t. and was evaporated. The residue was redissolved in ethyl acetate, washed with 1 N aqueous HCl (2×), saturated aqueous NaHCO₃ and brine, dried over MgSO₄ and evaporated.

General Procedure A3 (GP A3): Synthesis of Disubstituted Amines

To a stirred solution of 4.0 eq. of the amine in DMF was added dropwise a solution of 1.0 eq of methyl 4-[(bromoacetyl)amino]benzoate (Example IV) in DMF at room temperature. After stirring for 48 h, the solvent was evaporated. The residue was purified by flash chromatography.

### Methyl 4-({N-[2-(2-pyridinyl)ethyl]glycyl}amino)benzoate

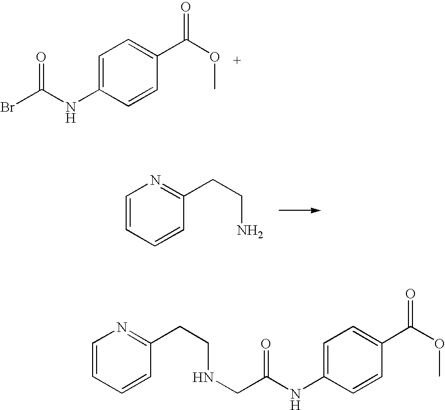

To a stirred solution of 2-(2-aminoethyl)pyridine (4.89 g, 40.0 mmol) in DMF (15 ml), was added dropwise, methyl 4-[(bromoacetyl)amino]benzoate (2.72 g, 10.0 mmol) in DMF (30 ml) at room temperature. After stirring for 48 h at room temperature, the solvent was evaporated. The residue was purified by flash chromatography (CH₂Cl₂/MeOH/NH₃ 9:0.05:0.1) affording 2.30 g (73%) of methyl 4-({N-[2-(2-pyridinyl)ethyl]glycyl}amino)benzoate as a yellow oil. TLC (CH₂Cl₂/MeOH/NH₃ 9:1:0.1): R_{f} 0.60; ¹H-NMR (500 MHz, D₆-DMSO) 10.10 (br s, 1H), 8.50-8.48 (m, 1H), 7.92 (d, J=8.8 Hz, 2H), 7.74-7.69 (m, 3H), 7.30 (d, J=7.8 Hz, 1H), 7.24-7.21 (m, 1H), 3.84 (s, 3H), 3.36 (s, 2H), 2.93-2.91 (m, 4H) (amine proton not observed); ESI-MS: [M+H]⁺=314.1.

TABLE 1
33/57 Characterization of reaction products according to Step A

| Example | | Procedure/ | | | | | | HPLC |
|---|---|---|---|---|---|---|---|---|
| No. | Structure | (Starting material) | Yield [%] | Product | M.p. [° C.] | ESI-MS | t_{R}[min] | |
| 1 | 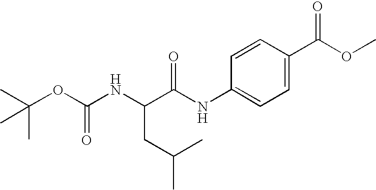 | GP A1 (Example 1) | 75 | white solid | 0.52 (CH₂Cl₂/MeOH 9:0.1) | 72-73 | 309.1 [M + H]⁺ | n.d. |
| 2 | 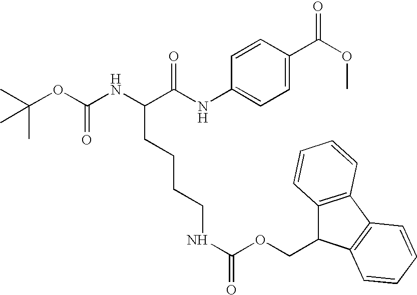 | GP A2 (methyl 4-aminobenzoate & N²BOC-N⁶-FMOC-L-lysine | 63 | white solid | 0.58 (CH₂Cl₂/MeOH 9:0.5) | 179-181 | 603 [M + H⁺ | n.d. |
| 3 | 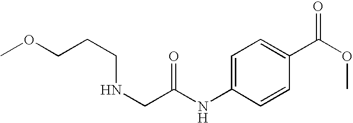 | as described in the precursor synthesis example IV | 65 | pale red solid | 0.36 (CH₂Cl₂/MeOH 9:1) | 49-50 | 281.0 [M + H]⁺ | n.d. |
| 4 | 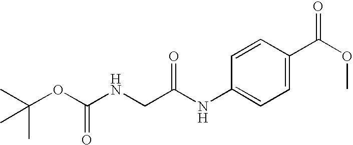 | GP A2, (methyl 4- aminobenzoate & BOC- glycine) trituration with ethyl acetate affords pure product | 55 | white solid | 0.64 (CH₂Cl₂/MeOH 9:1) | 140-142 | n.d. | n.d. |
| 5 | 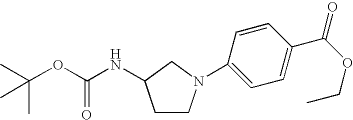 | same method as precursor synthesis: Example VIII (ethyl 4-bromobeuzoate & tert-butyl 3- pyrrolidinylcarbamate) | 30 | pale brown solid | 0.62 (CH₂Cl₂/MeOH 9:0.5) | 128-129 | 335.3 [M + H]⁺ LC- MS | 22.9 Method D |
| 6 | 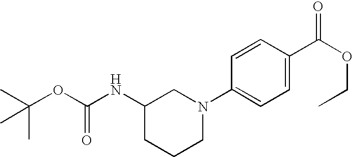 | precursor synthesis: Example VIII | 51 | pale yellow solid | 0.66 (CH₂Cl₂/MeOH 9:0.2) | 107-108 | 348 [M]⁺EI-MS | n.d. |
| 7 | 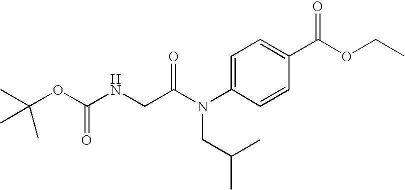 | Example IX, then GP A1 (Boc-glycin-N- carboxyanhydride) FC: CH₂Cl₂/MeOH 1:0-1:0.1, Purity: 40% by HPLC | 14 | pale brown foam | 0.48 (CH₂Cl₂/MeOH 9:0.2) | n.d. | 379.1 [M + H]⁺ LC- MS | 24.3 Method A |
| 8 | 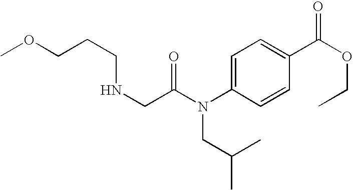 | precursor synthesis: Example IX, than same method as in precursor synthesis: Example IV (bromoacetyl bromide & methoxypropylamine) | 85 | pale yellow oil | 0.24 (CH₂Cl₂/MeOH/NH₃9:1:0.1) | n.d. | 351.5 [M + H]⁺ | 20.9 Method A |
| 9 | 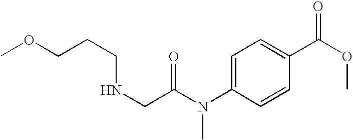 | same method as in example IV (methyl 4- (methylamino)benzoate & bromoacetyl bromide, then GP A3 (methoxypropylamine) | 93 | yellow solid | 0.52 (CH₂Cl₂/MeOH/NH₃9:1:0.1) | n.d. | 294 [M]⁺GC-MS (EI) | n.d. |
| 10 | 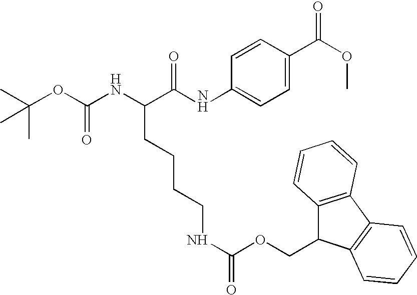 | GP A2 (methyl 4-aminobenzoate & N²-BOC-N⁶-FMOC-D-lysine) | 92 | white solid | 0.49 (CH₂Cl₂/MeOH 9:0.5) | 175-176 | 603 [M + H]⁺FAB-MS | 25.0 Method A |
| 11 | 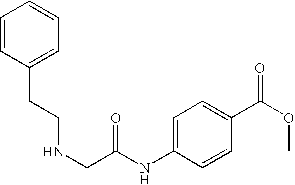 | GP A3, FC CH₂Cl₂/NH₃9:0.1 (2-phenylethylamine & methyl 4-[(bromoacetyl)- amino]benzoate) | 53 | yellow solid | 0.44 (CH₂Cl₂/MeOH 9:0.5) | n.d. | 313.1 [M + H]⁺ | 18.3 Method A |
| 12 | 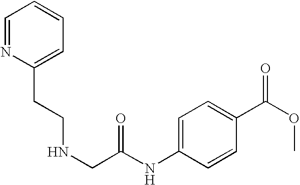 | GP A3, FC CH₂Cl₂/MeOH/NH₃9:0.05:0.1 (2-(2- pyridinyl)ethylamine & methyl 4-[(bromoacetyl)- amino]benzoate) | 73 | yellow oil | 0.60 (CH₂Cl₂/MeOH/NH₃9:1:0.1) | n.d. | 314.1 [M + H]⁺ | n.d. |
| 13 | 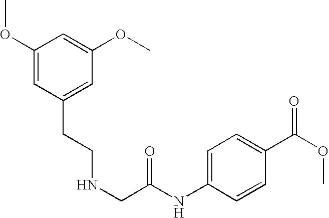 | GP A3, FC CH₂Cl₂/MeOH/NH₃9:0.05:0.1 (2-(3,5- dimethoxyphenyl)ethylamine & methyl 4-[(bromoacetyl)- amino]benzoate) | 95 | brown oil | 0.70 (CH₂Cl₂/MeOH 9:0.5) | n.d. | 373.0 [M + H]⁺ | n.d. |
| 14 | 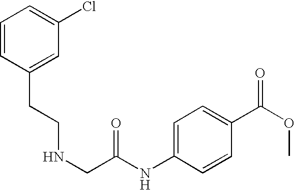 | GP A3, FC CH₂Cl₂/MeOH/NH₃9 9:0.05:0.1 (2-(3- chlorophenyl)ethylamine & methyl 4-[(bromoacetyl)- amino]benzoate) | 89 | brown oil | 0.34 (hexane/ethyl acetate) | n.d. | 347.0 [M + H]⁺ | 19.5 Method A |
| 15 | 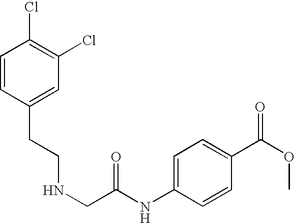 | GP A3, FC CH₂Cl₂/MeOH/NH₃9:0.01:0.1 (2-(3,4- dichlorophenyl)ethylamine & methyl 4-[(bromoacetyl)- amino]benzoate) | 96 | yellow oil | 0.48 (CH₂Cl₂/MeOH 9:0.5) | n.d. | 380.9 [M + H]⁺ | 20.1 Method A |
| 16 | 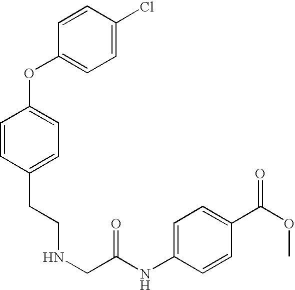 | GP A3, FC Cyclohexane/ Ethyl acetate 8:2, 7:3 (2-[4- (4-chlorophenoxy)phenyl]- ethylamine & methyl 4- [(bromoacetyl)amino]benzoate) | 61 | brown oil | 0.40 (CH₂Cl₂/MeOH/AcOH 9.5:0.5:0.1) | n.d. | 439.0 [M + H]⁺ | n.d. |

Step B
General Procedure B (GP B): Cleavage of the Boc-Protecting Group with Trifluoro-Acetic Acid

To a solution of the Boc-protected amine was added 20 vol % trifluoroacetic acid in dichloromethane at 0° C. Stirring was continued at room temperature for 0.5-24 h. The solvent was removed at room temperature under reduced pressure. The residue was coevaporated twice with dichloromethane, dried under high vacuum and subjected to the reaction step C without further purification.

Step C

General Procedure (GP Cl): Coupling of Amines with 2-{4-[(2-toluidinocarbonyl)-amino]phenyl}acetic acid:

A solution of 1.0 eq. 2-{4-[(2-toluidinocarbonyl)amino]phenyl}acetic acid, 1.1 eq. HOBt and 1.1 eq. EDCI in DMF was stirred for 2 h at r.t. After addition of 1.0 eq. amine e.g. as TFA salt and 3-9 eq. ethylisopropylamine stirring was continued for 18 h at r.t. The reaction mixture was poured into the 4-fold amount of water. The precipitate was collected by filtration, washed with cold water and dried in vacuum. If necessary the product was purified by trituration or by flash-chromatography.

### Methyl 4-([({4-[(2-toluidinocarbonyl)amino]phenyl}acetyl)L-leucin]amino)-benzoate

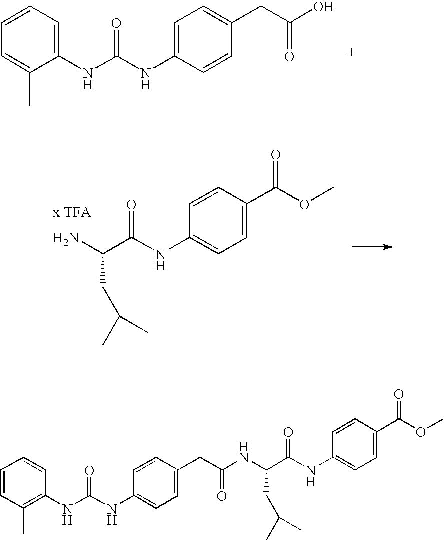

Methyl 4-[(L-leucin)amino]benzoate trifluoroacetate (3.81 g, 10.1 mmol) was reacted according to GP C1 in a total volume of 60 ml of dimethylacetamide. Trituration with CH₂Cl₂ yielded 4.78 g (90%) pale brown solid. M.p. 250-252° C., TLC (AcOH:MeOH:CH₂Cl₂ 0.1:0.5:9): R_{f} 0.46; ¹H-NMR (400 MHz, D₆-DMSO):10.47 (s, 1H), 8.96 (s, 1H), 8.39 (d, 7.7 Hz, 1H), 7.93-7.89 (m, 3H), 7.83 (d, 7.8 Hz, 1H), 7.75 (d, 8.8 Hz, 2H), 7.37 (d, 8.4 Hz, 2H), 7.18-7.12 (m, 4H), 6.95-6.92 (m, 1H), 4.49-4.43 (m, 1H), 3.82 (s, 3H), 3.47-3.38 (m, 2H), 2.24 (s, 3H), 1.66-1.50 (m, 3H), 0.92 (d, 6.4 Hz, 3H), 0.86 (d, 6.4 Hz, 3H); ESI-MS: 531.3 [M+H]⁺.

TABLE 2
The following examples were prepared by subsequently applying the general procedures B & C1/C2 as indicated.

| Ex- | | | | | | | | |
|---|---|---|---|---|---|---|---|---|
| am- | | Procedure/ | | | | | | |
| ple | | (Starting | Yield | | | | | HPLC |
| No. | Structure | material) | [%] | Product | Rᵣ | M.p.[° C.] | ESI-MS | t_{R}[min] |
| 17 | 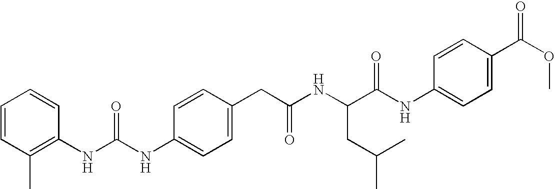 | 1) GP B 2) GP Cl, 9 eq. DIPEA/ (1 & example 1) | 90 | pale brown solid | 0.46 (CH₂Cl₂/MeOH/AcOH 9:0.5:0.1) | 250-252 | 531.3 [M + H]⁺ | 26.6 Method A |
| 18 | 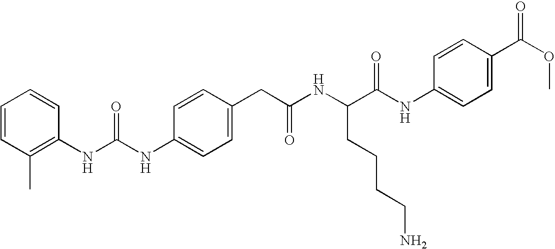 | 1) GP B 2) GP Cl, 9 eq. DIPEA (2 & example 1) FMOC is cleaved during step C1 | crude product was em ployed in next step | yellow solid | 0.86 (CH₂Cl₂/MeOH 9:1.5) | n.d. | 546 [M + H]⁺ | n.d. |
| 19 | 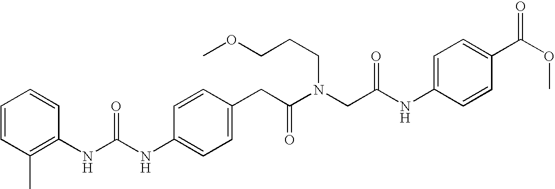 | 1) GP B 2) GP Cl, 3 eq. DIPEA (2 & example 1) | 64 | white solid | 0.50 (CH₂Cl₂/MeOH 9:1) | 197-198 | 547.0 [M + H]⁺ | 21.8 Method A |
| 20 | 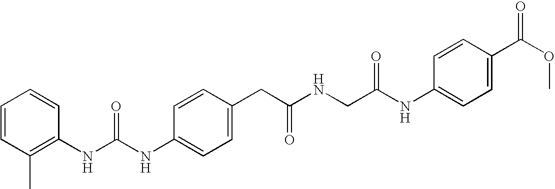 | 1) GP B 3) GP Cl, 9 eq DIPEA (4 & example 1) | 96 | pale brown solid | 0.30 (CH₂Cl₂/MeOH 9:1) | 248-250 | 515.5 [M + H]⁺ | 22.6 Method A |
| 21 | 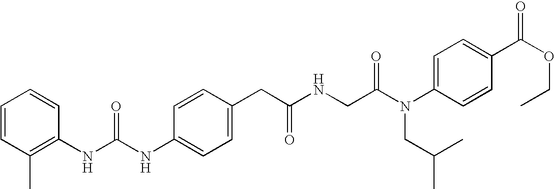 | 1) GP B 3) GP Cl, 9 eq DIPEA (7 & example 1) | 14 | pale yellow oil | 0.56 (CH₂Cl₂/MeOH 9:1) | — | 545.0 [M + H]⁺ | n.d. |
| 22 | 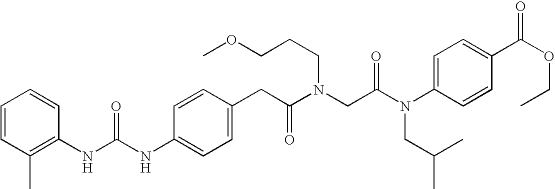 | GP Cl, 3 eq DIPEA (8 & example 1) | 43 | pale yellow solid | 0.82 (CH₂Cl₂/MeOH 9:1) | 123-125 | 617.6 [M + H]⁺ | n.d. |
| 23 | 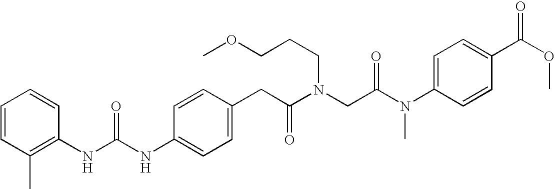 | GP Cl, 3 eq DIPEA (9 & example 1) | 43 | pale brown oil | 0.74 (CH₂Cl₂/MeOH 9:1) | n.d. | 561.2 [M + H]⁺ | n.d. |
| 24 | 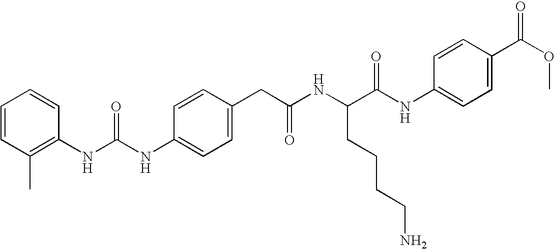 | 1) GP B 2) GP Cl, 9 eq DIPEA (10 & example 1), FMOC is cleaved during step C1 | crude product was em- ployed in next step | pale brown solid | 0.86 (CH₂Cl₂/MeOH 9:1.5) | n.d. | 546.5 [M + H]⁺LC-MS | n.d. |
| 25 | 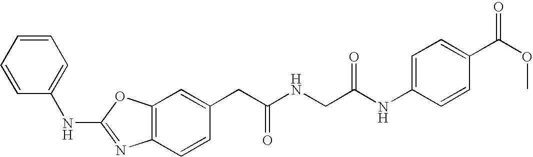 | 1) GP B 3) GP Cl, 3 eq DIPEA (4 & example X) | 79 | pale brown solid | 0.30 (CH₂Cl₂/MeOH 9:0.5) | 235-237 | 459.0 [M + H]⁺ | 20.3 Method A |
| 26 | 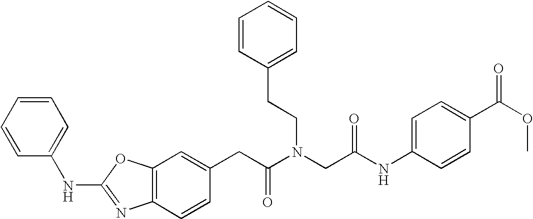 | GP Cl, 3 eq DIPEA (11 & example X) | 85 | white solid | 0.46 (CH₂Cl₂/MeOH 9:0.5) | 110-112 | 563.1 [M + H]⁺ | 23.2 Method A |
| 27 | 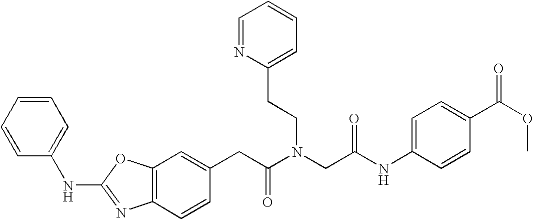 | GP Cl, 3 eq DIPEA (12 & example X) | 62 | white solid | 0.16 (CH₂Cl₂/MeOH 9:0.5) | 102-104 | 564.2 [M + H]⁺ | 20.0 Method A |
| 28 | 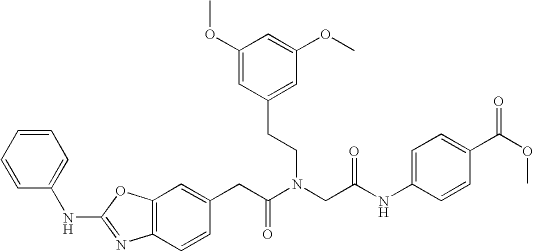 | GP Cl, 3 eq DIPEA (13 & example X) | 90 | pale brown solid | 0.82 (CH₂Cl₂/MeOH 9:1) | 111-113 | 623.1 [M + H]⁺ | 23.1 Method A |
| 29 | 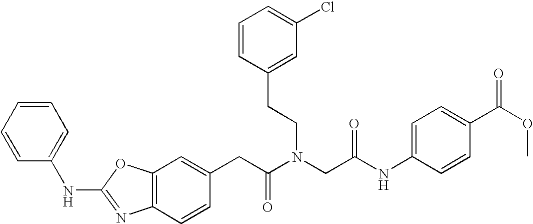 | GP Cl, 3 eq DIPEA (14 & example X) | 93 | pale yellow solid | 0.82 (CH₂Cl₂/MeOH 9:1) | 110-112 | 597.06 [M + H]⁺ | 23.7 Method A |
| 30 | 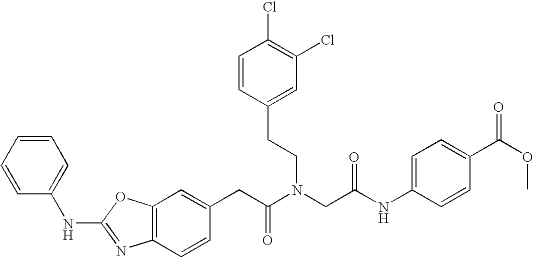 | GP Cl, 3 eq DIPEA (15 & example X) | 85 | pale yellow solid | 0.70 (CH₂Cl₂/MeOH/AcOH 9:1:0.1) | 89-91 | 631.0 [M + H]⁺ | 24.7 Method A |
| 31 | 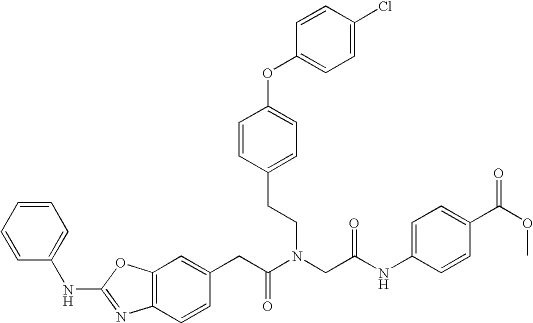 | GP Cl, 3 eq DIPEA (16 & example X) | 33 | white solid | 0.72 (CH₂Cl₂/MeOH 9:1) | 173-174 | 689.4 [M + H]⁺LC-MS | 21.9 Method A |
| 32 | 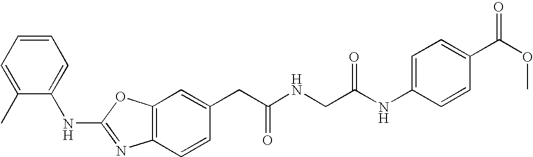 | GP Cl, 3 eq DIPEA (4 & example XI) | 78 | pale brown solid | 0.91 (CH₂Cl₂/MeOH/AcOH 9:1:0.1) | 156-158 | 473.3 [M + H]⁺LC-MS | 19.7 Method A |
| 33 | 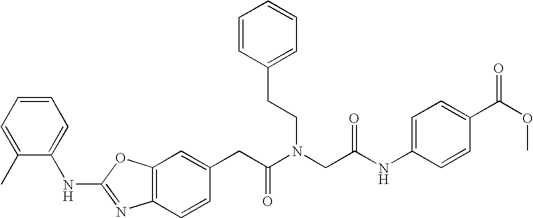 | GP Cl, 3 eq DIPEA (11 & example XI | 88 | white solid | 0.95 (CH₂Cl₂/MeOH/AcOH 9:1:0.1) | 100-105 | 577.1 [M + H]⁺ | 22.8 Method A |
| 34 | 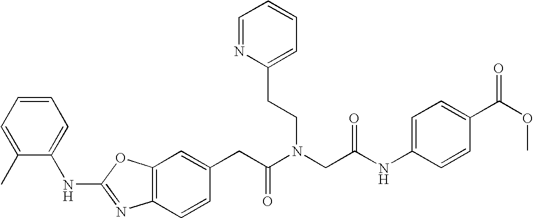 | GP Cl, 3 eq DIPEA (12 & example XI | 93 | yellow solid | 0.40 (CH₂Cl₂/MeOH 9:0.5) | 99-100 | 578.1 [M + H]⁺ | 19.5 Method A |
| 35 | 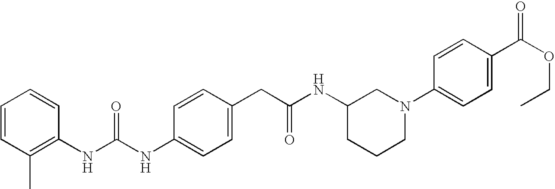 | 1) GP B 2) GP Cl, 9 eq DIPEA (6 & example 1) | 89 | pale brown solid | 0.46 (CH₂Cl₂/MeOH 9:1) | 225-227 | 475.04 [M + H]⁺ | 20.2 Method A |
| 36 | 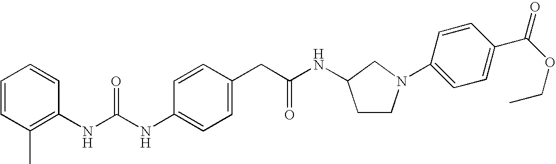 | 1) GP B 2) GP Cl, 9 eq DIPEA (5 & example 1) | 94 | white solid | 0.74 (CH₂Cl₂/MeOH 9:1) | 150-153 | 501.0 [M + H]⁺ | 22.7 Method A |

Step D
General Procedure D1 (GP DI): Ester Saponification

A solution or suspension of the ester and 1.1-10 eq eq. of KOH in water/ethanol or methanol and/or dioxane was stirred at 25-50° C. for 2-48 h. After washing with tert-butylmethylether (80 ml) the volume of the reaction mixture was reduced until a slight turbidity was observed. The solution was acidified to pH 2 by the addition of 1 N aqueous HCl. The precipitate was collected by filtration, washed with cold water and dried in vacuum.

General Procedure D2 (GP D2): Deprotection of Benzyl Esters/Benzyl Carbamates

A solution or suspension of the ester and 10% Pd—C (10%) in dimethylformamide was hydrogenated for 12 h at r.t. and 50 bar hydrogen pressure. The reaction mixture was filtered through celite. Evaporation of the filtrate and purification of the crude product by preparative HPLC (LiChrospher RP-18, 12 μM, 250×25 mm; flow rate 40 ml/min; eluent: acetonitrile/water mixture with 0.1% trifluoroacetic acid (vol./vol.), linear gradient of: 0 min. =40% acetonitrile, 20 min. =80% acetonitrile) afforded the product.

General Procedure D3 (GP D3): Deprotection of Benzyl Esters

A solution or suspension of the ester and 10% Pd—C (10%) in tetrahydrofurane was hydrogenated for 18 h at r.t. under atmospheric hydrogen pressure. The reaction mixture was filtered through celite. Evaporation of the filtrate afforded the product.

General Procedure D4 (GP D4): Deprotection of Tert-Butyl Esters

A solution of the ester in 20% trifluoroacetic acid in methylenechloride (v/v) was stirred at room temperature. The solvent was evaporated and the residue was dried in high vacuum. If necessary, the product was purified by trituration (e.g. in CH₂Cl₂/MeOH) or by flash chromatography.

General Procedure Solid Phase Compound Synthesis SPS1 (GP SPS1):

Synthesis Scheme SPS1:

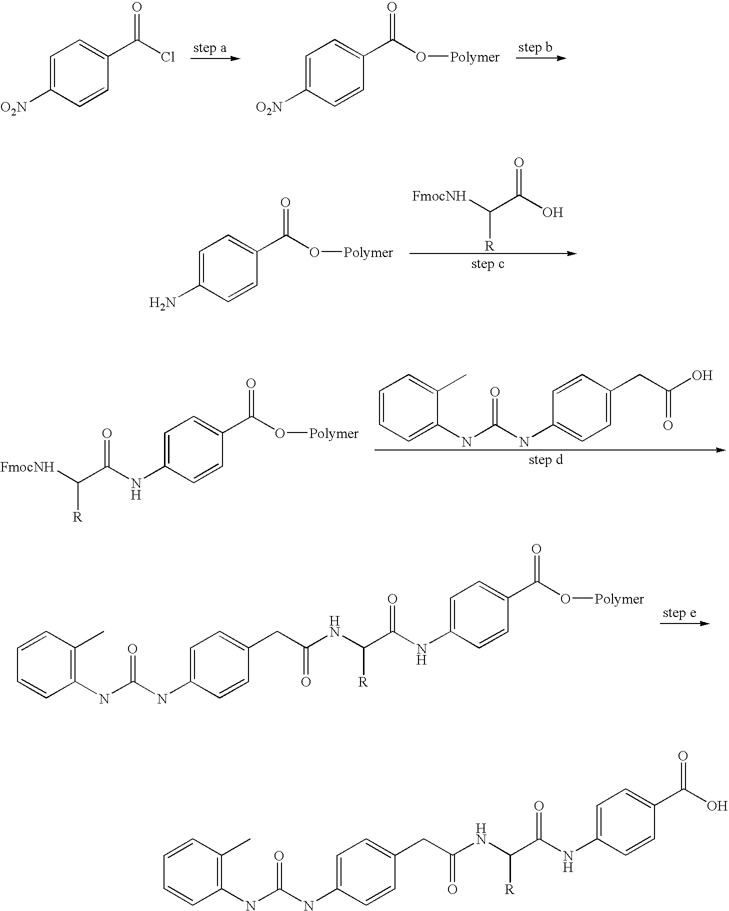

Step a: Wang polystyrene resin (1.5 g, Rapp-Polymere, Tübingen; loading 0.96 mmol/g) was swollen in tetrahydrofuran. The solvent was filtered off with suction and a solution of 737 mg diisopropyethylamine (737 mg) in tetrahydrofuran (4.5 ml) and a solution of 4-nitrobenzoic acidchloride (945 mg) in tetrahydrofuran (3.5 ml) was added. After shaking overnight at room temperature, the derivatized resin was subsequently washed with dimethylformamide, methanol, tetrahydrofuran and dichloromethane.

Step b: The derivatized resin was treated with a solution of tin(II) chloride dihydrate (2.7 g) in N-methylpyrrolidone (6 ml) and was shaken overnight at room temperature. The resin was subsequently washed with N-methylpyrrolidone, methanol, tetrahydrofuran and dichloromethane.

Step c: To a solution of the 9-fluorenylmethoxycarbonyl (Fmoc) protected amino acid (2.0 eq) in dimethylformamide (7 ml), O-(7-azabenzotriazol-1-yl)1,1,3,3-tetra-methyluronium hexafluorophosphate (1.06 g) and diisopropylethylamin (488 μl) were added. After shaking for 15 minutes, the derivatized resin was treated with this solution for 4 hours at room temperature. The derivatized resin was subsequently washed with dimethylformamide and tetrahydrofurane.

Step d: The derivatized resin was treated with 20% piperidine in dimethylformamide (15 ml, v/v) and was shaken at room temperature for 10 minutes. After washing 3 times with dimethylformamide, further 20% piperidine in dimethylformamide (15 ml, v/v) was added. After shaking for 20 minutes, the resin was subsequently washed with dimethylformamide and tetrahydrofurane. To a solution of 2-{4-[(2-toluidinocarbonyl)amino]phenyl}acetic acid (0.9 g, example I) in dimethylformamide (8 ml), O-(7-azabenzotriazol-1-yl)1,1,3,3-tetramethyluronium hexafluoro-phosphate (1.2 g) and diisopropylethylamin (557 μl) were added. After shaking the mixture for 15 minutes, the derivatized resin was treated with this solution for 4 hours at room temperature. The derivatized resin was subsequently washed with dimethylformamide and tetrahydrofurane.

Step e: For removal of the product from the resin, the derivatized resin was shaken with 10 ml of trifluoroacetic acid/dichloromethane 1:1 (v/v) for 1 hour and was filtered off. The filtrate was concentrated under reduced pressure and purified on silica gel.

General Procedure Solid Phase Compound Synthesis SPS2 (GP SPS2):

Synthesis Scheme SPS2:

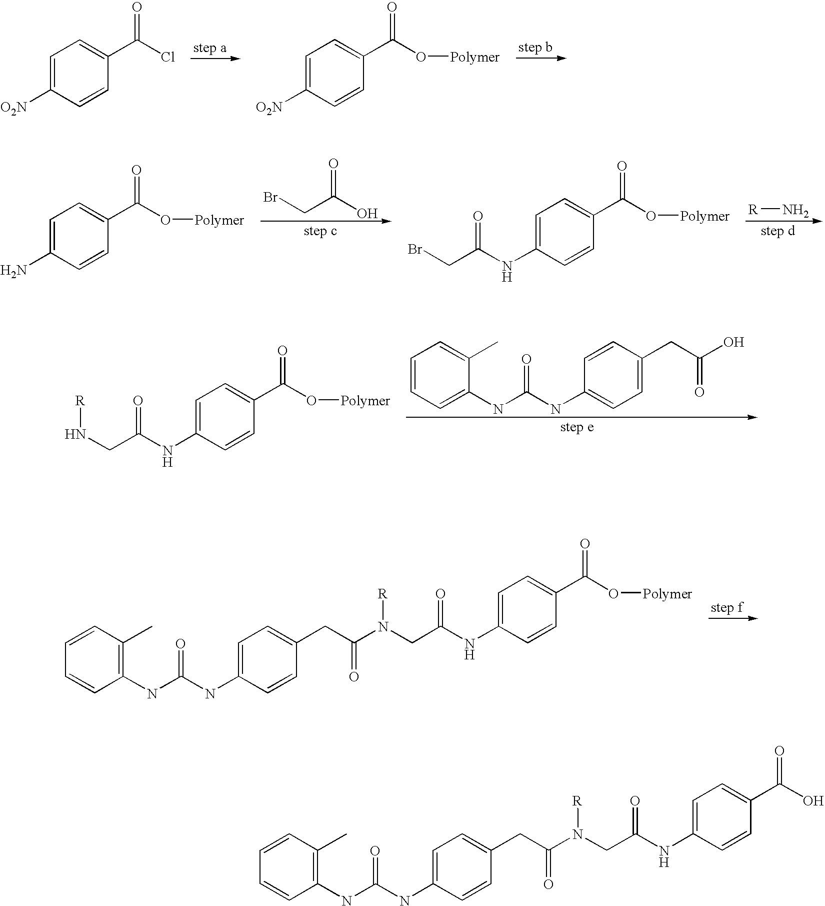

Step a: Wang polystyrene resin (1.5 g, Rapp-Polymere, Tübingen; loading 0.96 mmol/g) was swollen in tetrahydrofuran. The solvent was filtered off with suction and a solution of diisppropyethylamine (737 mg) in tetrahydrofuran (4.5 ml) and a solution of 4-nitrobenzoic acidchloride (945 mg) in tetrahydrofuran (3.5 ml) was added. After shaking overnight at room temperature, the derivatized resin was subsequently washed with dimethylformamide, methanol, tetrahydrofuran and dichloromethane.

Step b: The derivatized resin was treated with a solution of tin(II) chloride dihydrate (2.7 g) in N-methylpyrrolidone (6 ml) and was shaken overnight at room temperature. The resin was subsequently washed with N-methylpyrrolidone, methanol, tetrahydrofuran and dichloromethane.

Step c: A solution of bromoacetic acid (990 mg) in dimethylformamide (11 ml) was added to the derivatized resin. After shaking for 1 minute, a solution of diisopropyl-carbodiimide (1.26 g) in dimethylformamide (3 ml) was added. Following shaking over night, the derivatized resin was subsequently washed with dimethylformamide, methanol and dichloromethane.

Step d: A 1.8 molar solution of the amine derivative (8 ml) in dimethylformamide and diisopropylethylamine (0.8 g) was added to the derivatized resin. After shaking over night, the derivatized resin was subsequently washed with dimethylformamide, methanol and dichloromethane.

Step e: To a solution of 2-{4-[(2-toluidinocarbonyl)amino]phenyl}acetic acid (0.9 g, example I) in dimethylformamide (8 ml), O-(7-azabenzotriazol-1-yl)1,1,3,3-tetra-methyluronium hexafluorophosphate (1.2 g) and diisopropylethylamin (557 μl) were added. After shaking the mixture for 15 minutes, the derivatized resin was treated with this solution for 4 hours at room temperature. The derivatized resin was washed with dimethylformamide and tetrahydrofurane.

Step f: For removal of the product from the resin, the derivatized resin was shaken with 10 ml of trifluoroacetic acid/dichloromethane 1:1 (v/v) for 1 hour and was filtered off. The filtrate was concentrated under reduced pressure and purified on silica gel.

General Procedure Solid Phase Compound Synthesis SPS3 (GP SPS3):

Synthesis Scheme SPS3:

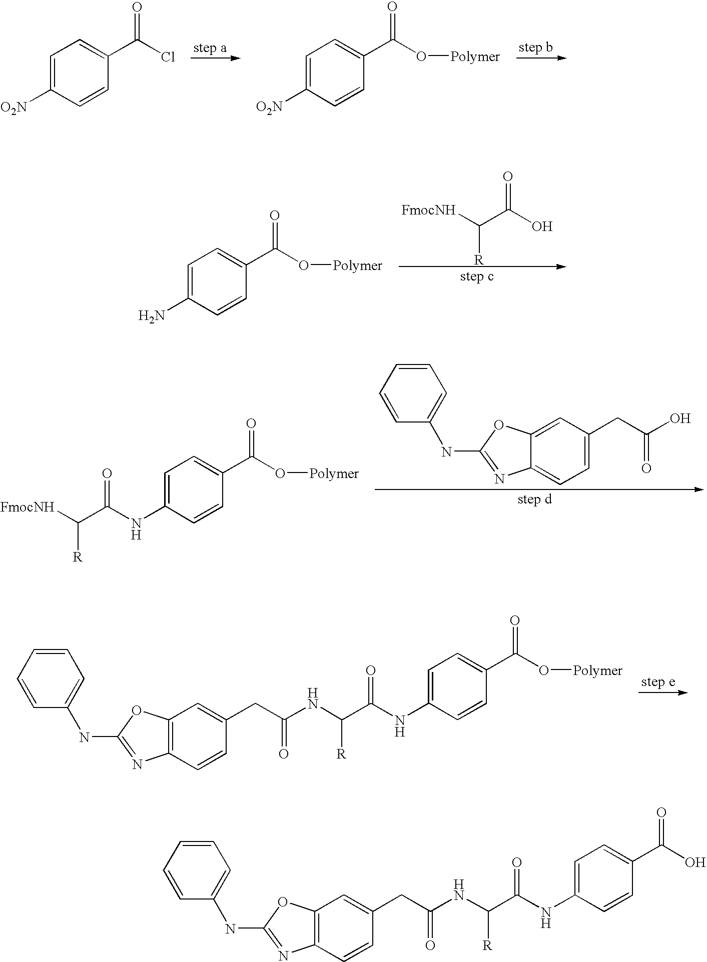

Step a: Wang polystyrene resin (1.5 g, Rapp-Polymere, Tübingen; loading 0.96 mmol/g) was swollen in tetrahydrofuran. The solvent was filtered off with suction and a solution of 737 mg diisopropyethylamine (737 mg) in tetrahydrofuran (4.5 ml) and a solution of 4-nitrobenzoic acidchloride (945 mg) in tetrahydrofuran (3.5 ml) was added. After shaking overnight at room temperature, the derivatized resin was subsequently washed with dimethylformamide, methanol, tetrahydrofuran and dichloromethane.

Step b: The derivatized resin was treated with a solution of tin(II) chloride dihydrate (2.7 g) in N-methylpyrrolidone (6 ml) and was shaken overnight at room temperature. The resin was subsequently washed with N-methylpyrrolidone, methanol, tetrahydrofuran and dichloromethane.

Step c: To a solution of the 9-fluorenylmethoxycarbonyl (Fmoc) protected amino acid (2.0 eq) in dimethylformamide (7 ml), O-(7-azabenzotriazol-1-yl)1,1,3,3-tetra-methyluronium hexafluorophosphate (1.06 g) and diisopropylethylamin (488 μl) were added. After shaking for 15 minutes, the derivatized resin was treated with this solution for 4 hours at room temperature. The derivatized resin was subsequently washed with dimethylformamide and tetrahydrofurane.

Step d: The derivatized resin was treated with 20% piperidine in dimethylformamide (15 ml, v/v) and was shaken at room temperature for 10 minutes. After washing 3 times with dimethylformamide, further 20% piperidine in dimethylformamide (15 ml, v/v) was added. After shaking for 20 minutes, the resin was subsequently washed with dimethylformamide and tetrahydrofurane. To a solution of (2-anilino-1,3-benzoxazol-6-yl)acetic acid (0.9 g, example X) in dimethylformamide (8 ml), 0-(7-azabenzotriazol-1-yl)1,1,3,3-tetramethyluronium hexafluorophosphate (1.2 g) and diisopropylethylamin (557 μl) were added. After shaking the mixture for 15 minutes, the derivatized resin was treated with this solution for 4 hours at room temperature. The derivatized resin was subsequently washed with dimethylformamide and tetrahydrofurane.

Step e: For removal of the product from the resin, the derivatized resin was shaken with 10 ml of trifluoroacetic acid/dichloromethane 1:1 (v/v) for 1 hour and was filtered off. The filtrate was concentrated under reduced pressure and purified on silica gel.

General Procedure Solid Phase Compound Synthesis SPS4 (GP SPS4):

Synthesis Scheme SPS4:

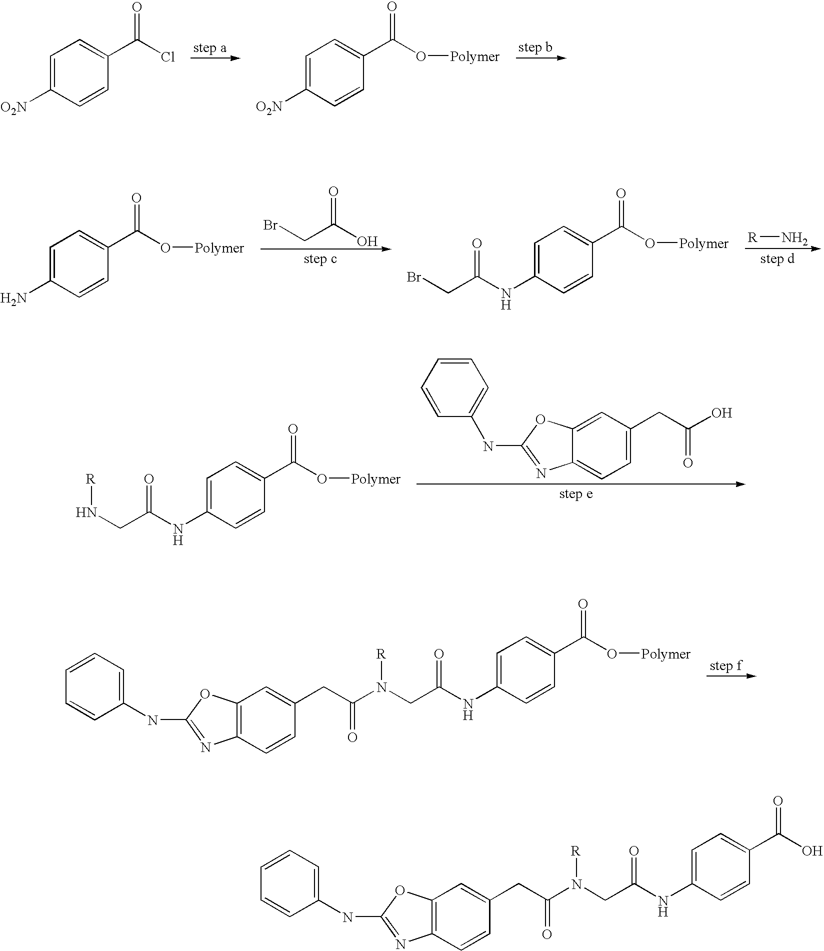

Step a: Wang polystyrene resin (1.5 g, Rapp-Polymere, Tübingen; loading 0.96 mmol/g) was swollen in tetrahydrofuran. The solvent was filtered off with suction and a solution of diisopropyethylamine (737 mg) in tetrahydrofuran (4.5 ml) and a solution of 4-nitrobenzoic acidchloride (945 mg) in tetrahydrofuran (3.5 ml) was added. After shaking overnight at room temperature, the derivatized resin was subsequently washed with dimethylformamide, methanol, tetrahydrofuran and dichloromethane.

Step b: The derivatized resin was treated with a solution of tin(II) chloride dihydrate (2.7 g) in N-methylpyrrolidone (6 ml) and was shaken overnight at room temperature. The resin was subsequently washed with N-methylpyrrolidone, methanol, tetrahydrofuran and dichloromethane.

Step c: A solution of bromoacetic acid (990 mg) in dimethylformamide (11 ml) was added to the derivatized resin. After shaking for 1 minute, a solution of diisopropylcarbodiimide (1.26 g) in dimethylformamide (3 ml) was added. Following shaking over night, the derivatized resin was subsequently washed with dimethyl-formamide, methanol and dichloromethane.

Step d: A 1.8 molar solution of the amine derivative (8 ml) in dimethylformamide and diisopropylethylamine (0.8 g) was added to the derivatized resin. After shaking over night, the derivatized resin was subsequently washed with dimethylformamide, methanol and dichloromethane.

Step e: To a solution of (2-anilino-1,3-benzoxazol-6-yl)acetic acid (0.9 g, example X) in dimethylformamide (8 ml), O-(7-azabenzotriazol-1-yl), 1,3,3-tetramethyluronium hexafluorophosphate (1.2 g) and diisopropylethylamin (557 μl) were added. After shaking the mixture for 15 minutes, the derivatized resin was treated with this solution for 4 hours at room temperature. The derivatized resin was washed with dimethylformamide and tetrahydrofurane.

Step f: For removal of the product from the resin, the derivatized resin was shaken with 10 ml of trifluoroacetic acid/dichloromethane 1:1 (v/v) for 1 hour and was filtered off. The filtrate was concentrated under reduced pressure and purified on silica gel.

TABLE 3
The following examples were prepared according to the general procedures D1-D4, SPS 1-4:

| | | Procedure/ | | | | | | |
|---|---|---|---|---|---|---|---|---|
| Example | | (Starting | Yield | | | | | |
| No. | Structure | material) | [%] | Product | R_{f} | M.p. [° C.] | ESI-MS | HPLC t_{R}[min] |
| 37 | 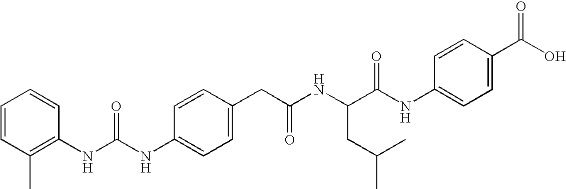 | GP D1; 10 eq. KOH (17) | 90 | white solid | 0.24 (CH₂Cl₂/MeOH/Ac OH 9:1:0.1) | 219-223 | 517.0 [M + H]⁺ | 21.3 Method A |
| 38 | 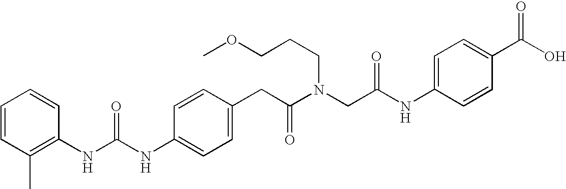 | GP D1; 1.1 eq. KOH (19) | 76 | white solid | 0.48 (CH₂Cl₂/MeOH/AcOH 9:1:0.1) | 210-218 | 570.8 [M + K]⁺ | 20.0 Method A |
| 39 | 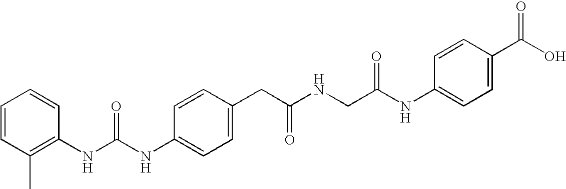 | GP D1, 1.1 eq. KOH (20) | 87 | pale brown solid | 0.24 (CH₂Cl₂MeOH/AcOH 9:1:0.1) | 268-271 | 460.9 [M + H]⁺ | 18.2 Method A |
| 40 | 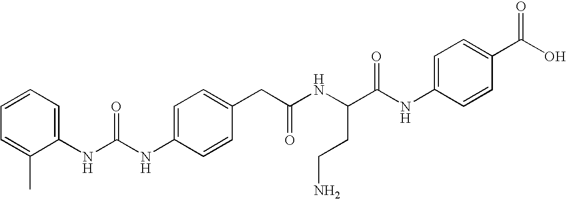 | SPS1 | n.d. | n.d. | n.d. | n.d. | 504.2 [M + H]⁺ | 5.7 Method C |
| 41 | 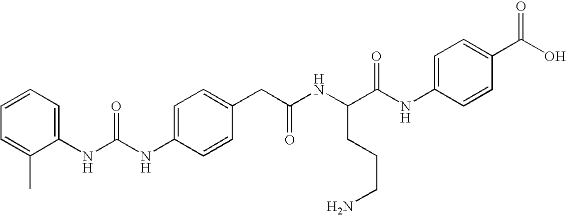 | SPS1 | n.d. | n.d. | n.d. | n.d. | 518.2 [M + H]⁺ | 5.7 Method C |
| 42 | 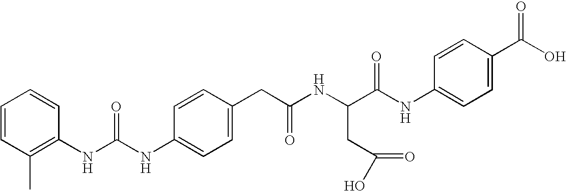 | SPS1 | n.d. | n.d. | n.d. | n.d. | 519.1 [M + H]⁺ | 6.5 Method C |
| 43 | 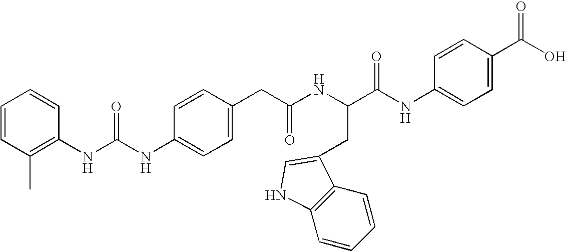 | SPS1 | n.d. | n.d. | n.d. | n.d. | 590.2 [M + H]⁺ | 8.5 Method C |
| 44 | 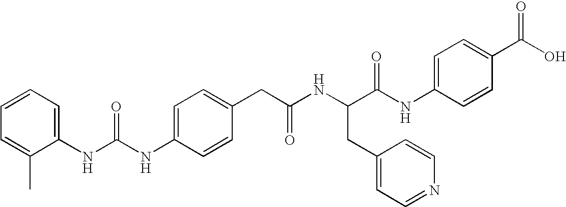 | SPS1 | n.d. | n.d. | n.d. | n.d. | 552.2 [M + H]⁺ | 6.0 Method C |
| 45 | 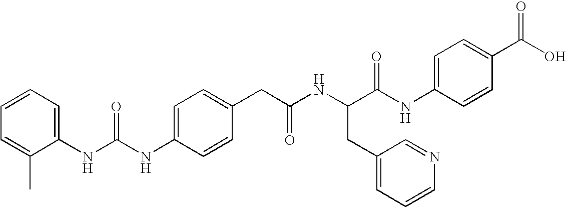 | SPS1 | n.d. | n.d. | n.d. | n.d. | 552.1 [M + H]⁺ | 6.1 Method C |
| 46 | 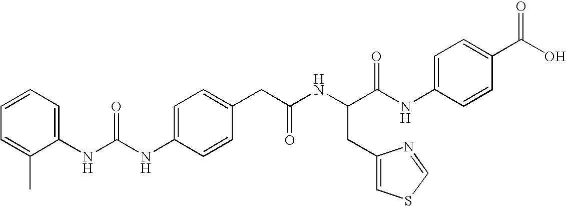 | SPS1 | n.d. | n.d. | n.d. | n.d. | 558.1 [M + H]⁺ | 7.3 Method C |
| 47 | 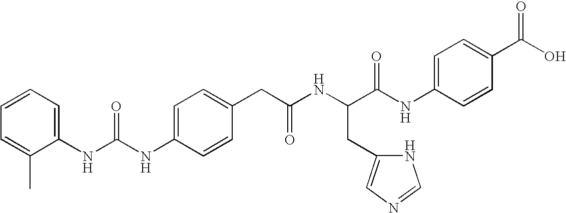 | SPSl | n.d. | n.d. | n.d. | n.d. | 541.2 [M + H]⁺ | 5.9 Method C |
| 48 | 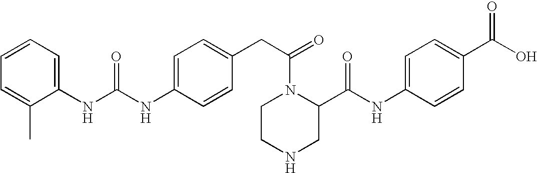 | SPS1 | n.d. | n.d. | n.d. | n.d. | 516.2 [M + H]⁺ | 5.9 Method C |
| 49 | 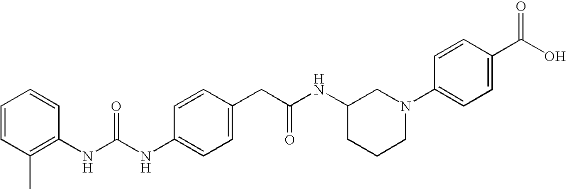 | GP D1, 1.1 eq. KOH (35) | 36 | white solid | 0.44 (CH₂Cl₂/MeOH 9:1) | 236-238 | 487.0 [M + H]⁺ | 19.8 Method A |
| 50 | 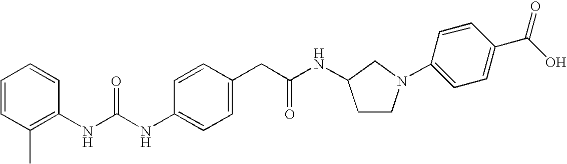 | GP D1, 1.1 eq. KOH (36) | 8 | pale brown solid | 0.42 (CH₂Cl₂/MeOH 9:1) | 239-242 | 473.0 [M + H]⁺ | 19.4 Method A |
| 51 | 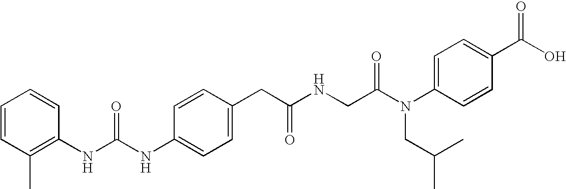 | GP D1, 1.1 eq. KOH (21) | 64 | white solid | 0.70 (CH₂Cl₂/MeOH/AcOH 9:1:0.1) | 131-132 | 517.3 [M + H]⁺ | 20.6 Method A |
| 52 | 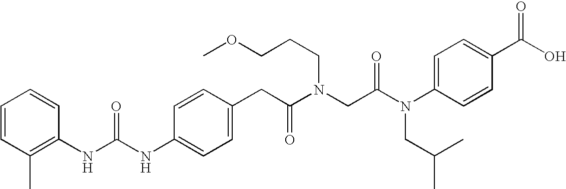 | GP D1, 1.1 eq. KOH (22) | 96 | white solid | 0.12 (CH₂Cl₂/MeOH/AcOH 9.5:0.5:0.1) | 76.0-76.5 | 589.3 [M + H⁺ | 20.9 Method A |
| 53 | 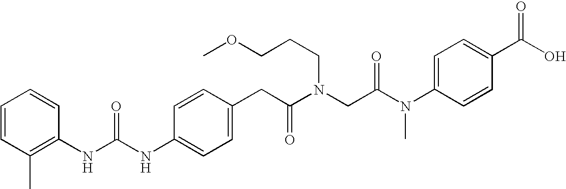 | GP D1, 1.1 eq. KOH (23) | 77 | pale yellow solid | 0.08 (CH₂Cl₂/MeOH/AcOH 9.5:0.5:0.1) | 63-64 | 547.2 [M + H]⁺ | 19.2 Method A |
| 53a | 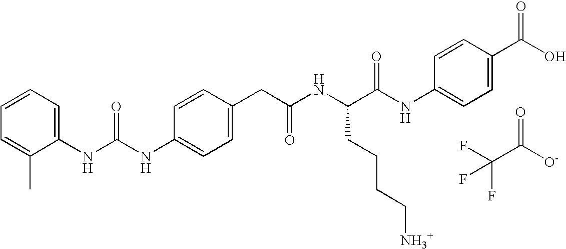 | GP D2 (18) | 3 | white solid | 0.05 (CH₂Cl₂/MeOH/AcOH 9:1:0.1) | 168-169 | 532.1 [M + H]⁺LC-MS | n.d. |
| 54 | 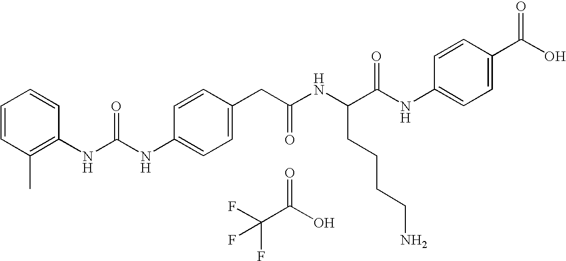 | GP D1, 4 eq. KOH (24) | 45 | white solid | 0.06 (CH₂Cl₂/MeOH 9:15) | 73-75 | 532.3 [M + H]⁺ | 17.7 Method A |
| 55 | 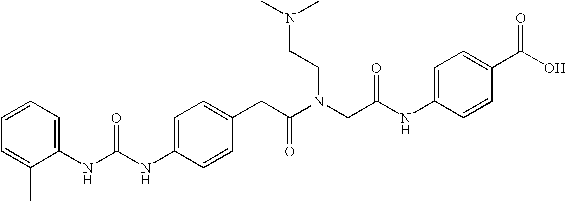 | SPS2 | n.d. | n.d. | n.d. | n.d. | 532.2 [M + H]⁺ | 6.2 Method C |
| 56 | 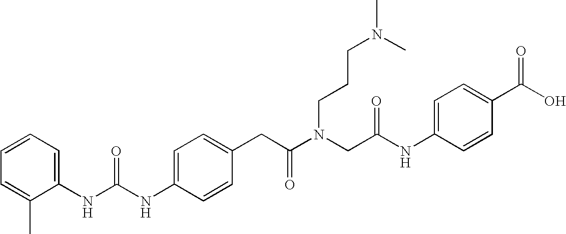 | SPS2 | n.d. | n.d. | n.d. | n.d. | 546.2 [M + H]⁺ | 5.9 Method C |
| 57 | 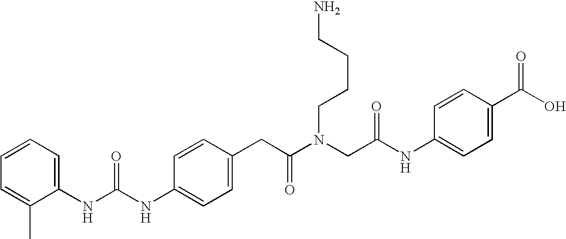 | SPS2 | n.d. | n.d. | n.d. | n.d. | 572.3 [M + ACN]⁺ | 5.7 Method C |
| 58 | 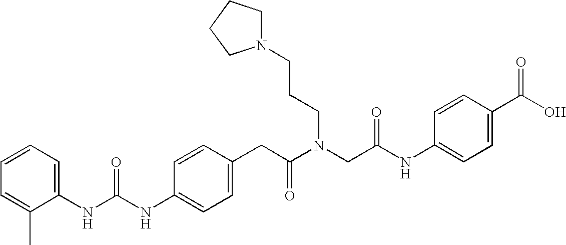 | SPS2 | n.d. | n.d. | n.d. | n.d. | 572.3 [M + H]⁺ | 6.3 Method C |
| 59 | 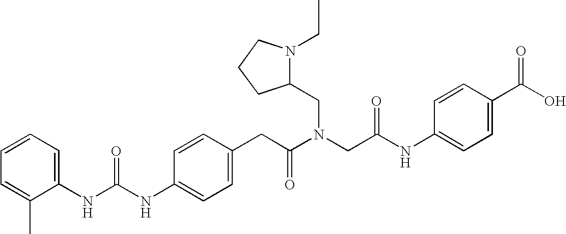 | SPS2 | n.d. | n.d. | n.d. | n.d. | 572.3 [M + H]⁺ | 6.6 Method C |
| 60 | 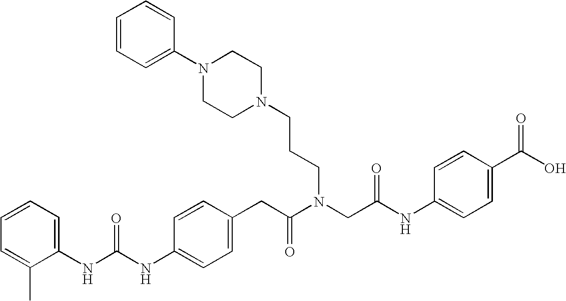 | SPS2 | n.d. | n.d. | n.d. | n.d. | 663.3 [M + H]⁺ | 7.6 Method C |
| 61 | 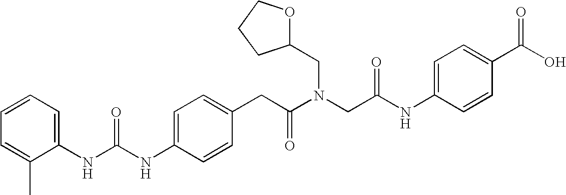 | SPS2 | n.d. | n.d. | n.d. | n.d. | 545.1 [M + H]⁺ | 7.9 Method C |
| 62 | 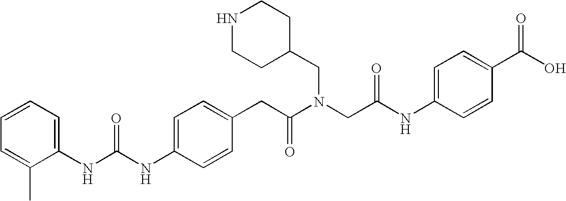 | SPS2 | n.d. | n.d. | n.d. | n.d. | 558.2 [M + H]⁺ | 5.7 Method C |
| 63 | 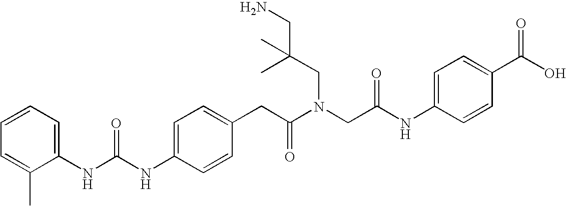 | SPS2 | n.d. | n.d. | n.d. | n.d. | 546.2 [M + H]⁺ | 6.1 Method C |
| 64 | 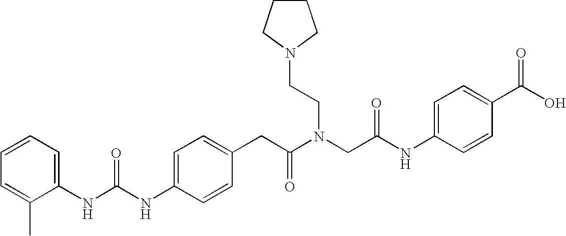 | SPS2 | n.d. | n.d. | n.d. | n.d. | 558.2 [M + H]⁺ | 6.0 Method C |
| 65 | 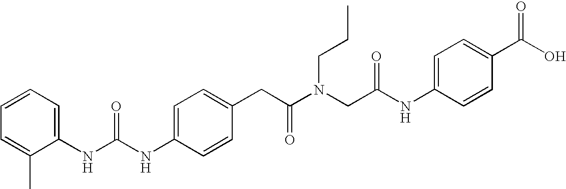 | SPS2 | n.d. | n.d. | n.d. | n.d. | 503.1 [M + H]⁺ | 8.0 Method C |
| 66 | 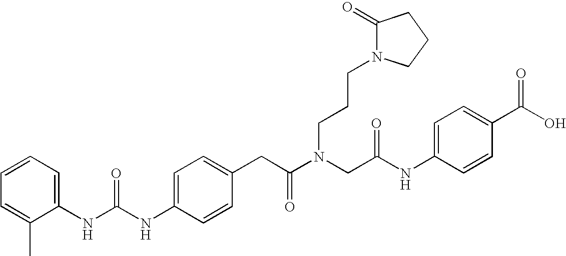 | SPS2 | n.d. | n.d. | n.d. | n.d. | 586.2 [M + H]⁺ | 7.1 Method C |
| 67 | 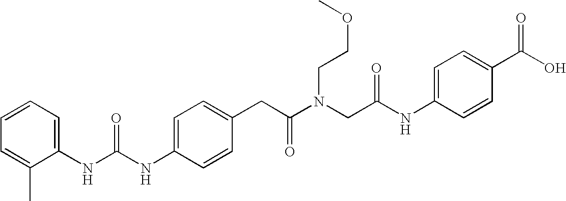 | SPS2 | n.d. | n.d. | n.d. | n.d. | 519.1 [M + H]⁺ | 7.4 Method C |
| 68 | 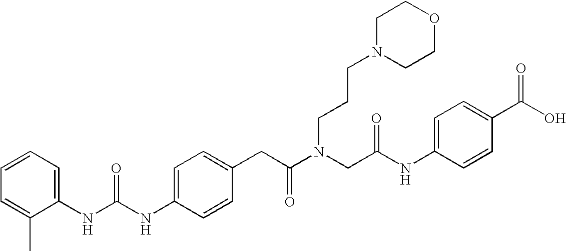 | SPS2 | n.d. | n.d. | n.d. | n.d. | 588.2 [M + H]⁺ | 5.8 Method C |
| 69 | 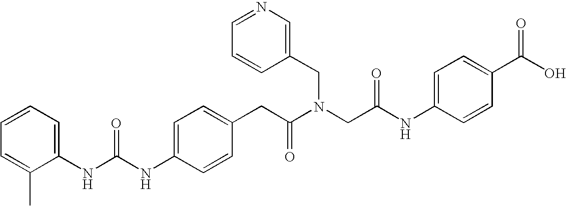 | SPS2 | n.d. | n.d. | n.d. | n.d. | 552.1 [M + H]⁺ | 5.9 Method C |
| 70 | 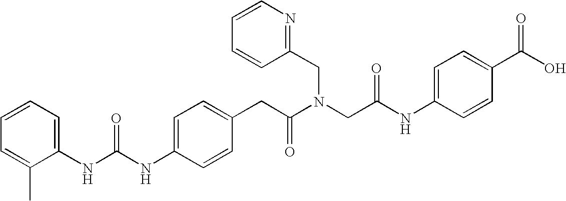 | SPS2 | n.d. | n.d. | n.d. | n.d. | 552.1 [M + H]⁺ | 6.2 Method C |
| 71 | 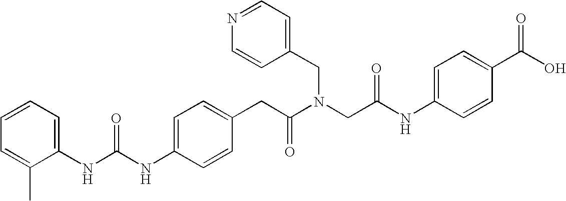 | SFS2 | n.d. | n.d. | n.d. | n.d. | 552.1 [M + H]⁺ | 5.8 Method C |
| 72 | 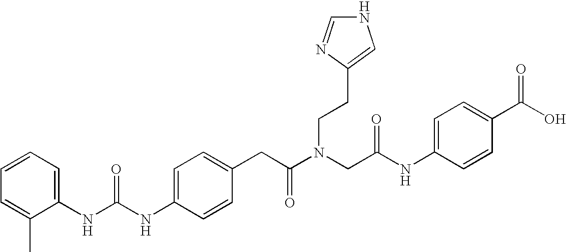 | SPS2 | n.d. | n.d. | n.d. | n.d. | 555.2 [M + H]⁺ | 5.7 Method C |
| 73 | 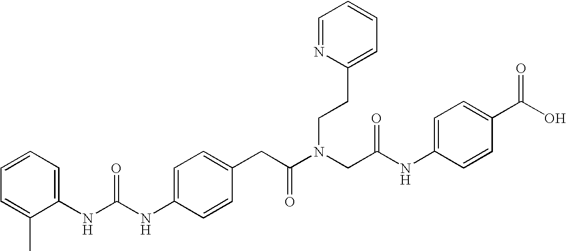 | SPS2 | n.d. | n.d. | n.d. | n.d. | 566.1 [M + H]⁺ | 6.0 Method C |
| 74 | 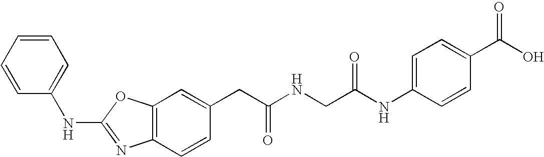 | GP D1; 2.1 eq KOH (25) | 85 | white solid | 0.06 (CH₂Cl₂/MeOH/AcOH 9:1:0.1) | >280 | 445.0 [M + H]⁺ | 18.6 Method D |
| 75 | 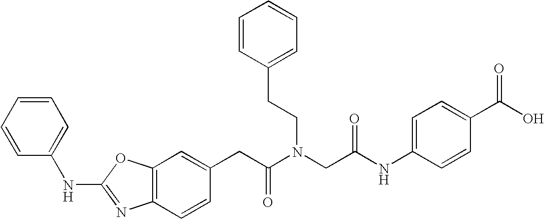 | GP D1; 1.1 eq KOH (26) | 62 | white solid | 0.40 (CH₂Cl₂/MeOH/AcOH 9:0.5:0.1) | 160-162 | 549.1 [M + H]⁺ | 21.6 Method A |
| 76 | 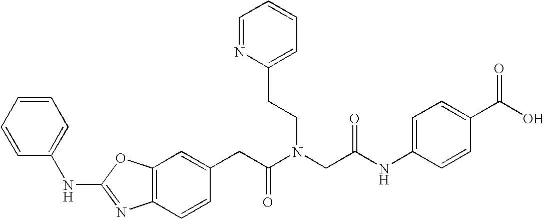 | GP D1; 2.1 eq KOH (27) | 98 | white solid | 0.08 (CH₂Cl₂/MeOH/AcOH 9:0.5:0.1) | 252-253 | 550.1 [M + H]⁺ | 18.0 Method A |
| 77 | 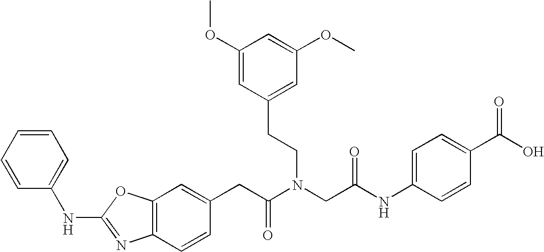 | GP D1; 1.1 eq KOH (28) | 66 | whit solid | 0.32 (CH₂Cl₂/MeOH/AcOH 9:0.5:0.1) | 130-13 | 609.1 [M + H]⁺ | 20.5 Method D |
| 78 | 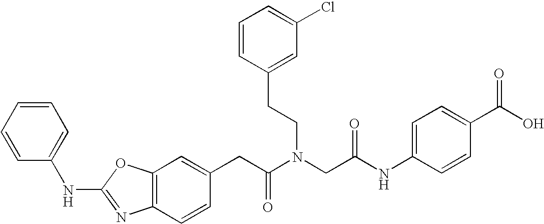 | GP D1; 1.1 eq KOH (29) | 96 | white solid | 0.32 (CH₂Cl₂/MeOH/AcOH 9:0.5:0.1) | 162-165 | 583.0 [M + H]⁺ | 22.5 Method A |
| 79 | 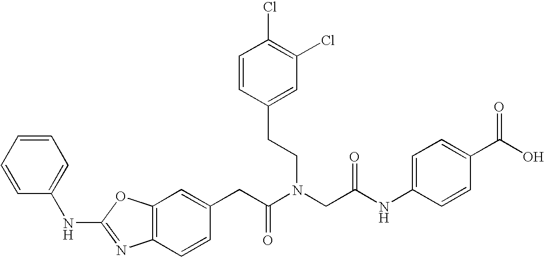 | GP D1; 1.1 eq KOH (30) | 79 | pale brown solid | 0.26 (CH₂Cl₂/MeOH/AcOH 9:0.5:0.1) | 170-171 | 616.9 [M + H]⁺ | 23.2 Method A |
| 80 | 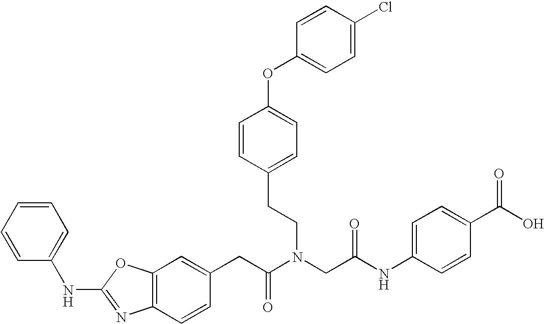 | GP D1; 1.1 eq KOH (31) | 89 | pale brown solid | 0.56 (CH₂Cl₂/MeOH/AcOH 9:0.5:0.1) | 156-158 | 675.0 [M + H]⁺ | 23.4 Method D |
| 81 | 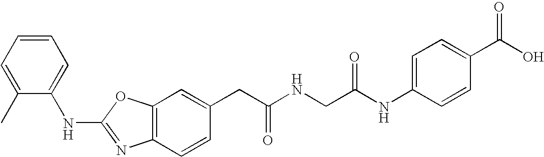 | GP D1; 2 eq KOH (32) | 89 | pale red | 0.16 (CH₂Cl₂/MeOH/AcOH 9:0.5:0.1) | 267-268 | 459.0 [M + H]⁺ | 16.4 Method A |
| 82 | 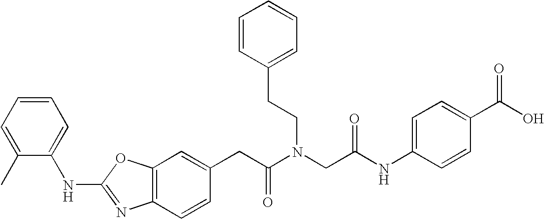 | GP D1; 2 eq KOH (33) | 86 | white solid | 0.32 (CH₂Cl₂/MeOH/AcOH 9:0.5:0.1) | 232-233 | 563.0 [M + H]⁺ | 19.9 Method D |
| 83 | 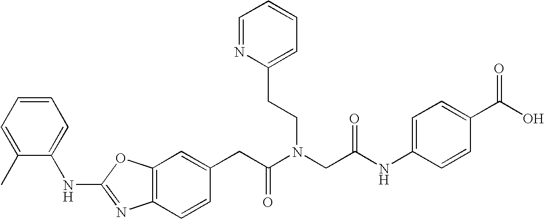 | GP D1; 2 eq KOH (34) | 30 | white solid | 0.18 (CH₂Cl₂/MeOH/AcOH 9:0.5:0.1) | 175-176 | 546.8 [M + H]⁺ | 16.4 Method D |
| 84 | 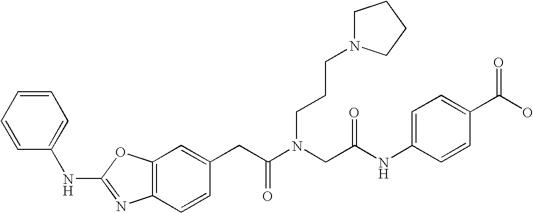 | GP SPS4 | n.d. | n.d. | n.d. | n.d. | 556.36 [M + H]⁺ | 6.1 Method C |
| 85 | 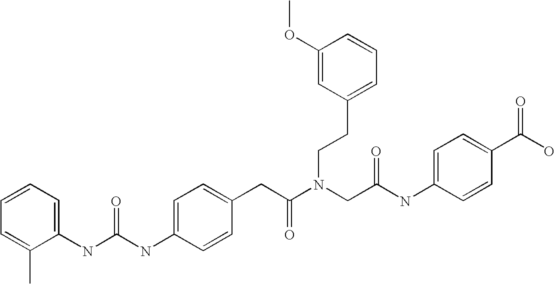 | GP SPS2 | n.d. | n.d. | n.d. | n.d. | 593.36 [M − H]⁺ | 9.1 Method C |
| 86 | 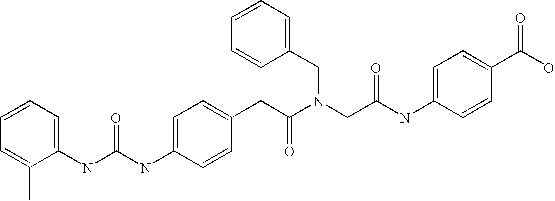 | GP SPS2 | n.d. | n.d. | n.d. | n.d. | 549.34 [M − H]⁺ | 8.8 Method C |
| 87 | 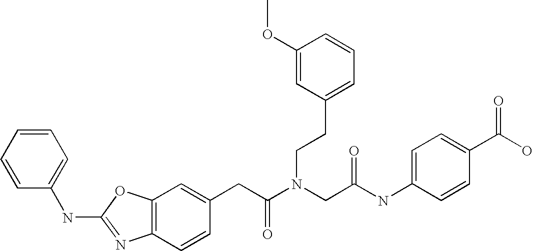 | GP SFS4 | n.d. | n.d. | n.d. | n.d. | 579.38 [M + H]⁺ | 9.1 Method C |
| 88 | 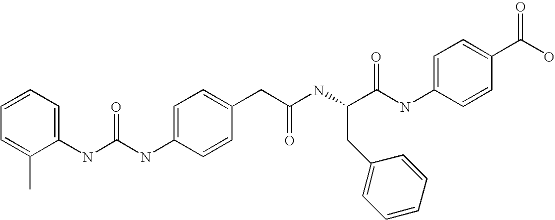 | GP SPS1 | n.d. | n.d. | n.d. | n.d. | 551.21 [M + H]⁺ | 8.7 Method C |
| 89 | 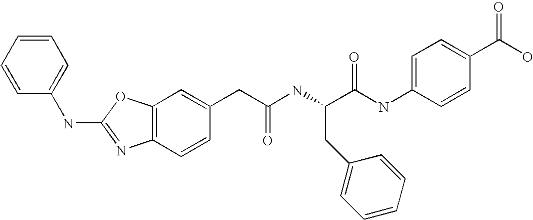 | GP SFS3 | n.d. | n.d. | n.d. | n.d. | 535.22 [M + H]⁺ | 8.8 Method C |
| 90 | 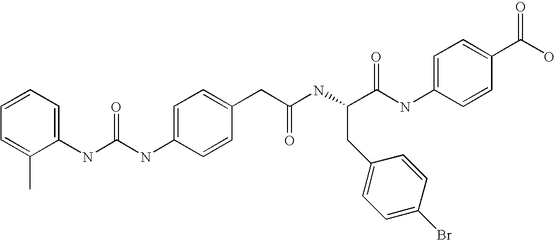 | GP SF51 | n.d. | n.d. | n.d. | n.d. | 630.19 [M + H]⁺ | 9.3 Method C |
| 91 | 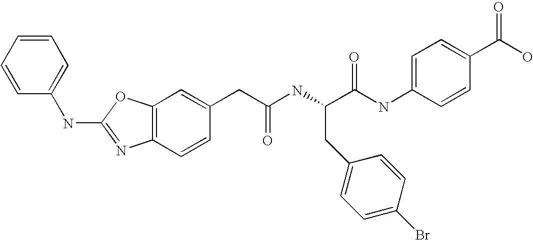 | GP SPS3 | n.d. | n.d. | n.d. | n.d. | 613.18 [M + H]⁺ | 9.4 Method C |
| 92 | 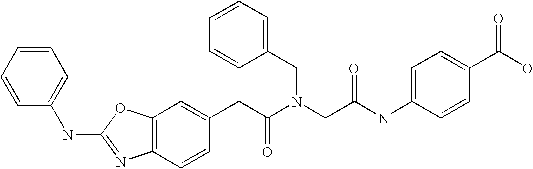 | GP SPS4 | n.d. | n.d. | n.d. | n.d. | 535.35 [M + H]⁺ | 8.8 Method C |

In Vitro Assay: Adhesion of Ramos Cells to Immobilized VCAM-1 (Domains 1-3)
Preparation of VCAM-1 (Extracellular Domains 1-3)

Complementary DNA (cDNA) encoding 7-domain form of VCAM-1 (GenBank accession #M60335) was obtained using Rapid-Screen™ cDNA library panels (OriGene Technologies, Inc) at Takara Gene Analysis Center (Shiga, Japan). The primers used were 5′-CCA AGG CAG AGT ACG CAA AC-3′ (sense) and 5′-TGG CAG GTA TTA TTA AGG AG-3′ (antisense). PCR amplification of the 3-domain VCAM-1 cDNA was perform using Pfu DNA polymerase (Stratagene) with the following sets of primers: (U-VCAMd1-3) 5′-CCA TAT GGT ACC TGA TCA ATT TAA AAT CGA GAC CAC CCC AGA A-3′; (L-VCAMdl-3) 5-CCA TAT AGC AAT CCT AGG TCC AGG GGA GAT CTC AAC AGT AAA-3′. PCR cycle was 94° C. for 45 sec, 55° C. for 45 sec, 72° C. for 2 min, repeating 15 cycles. After the purification of the PCR product, the fragment was digested with KpnI-AvrII. The digested fragment was ligated into pBluescript IISK(−) (Strategene), which was linearized by digesting with KpnI-XhoI. The ligation was followed by transformation to a Dam/Dcm methylase-free E. coli strain SCS110 (Strategene) to create the donor plasmid pHH7. To direct VCAM-1 molecule into the insect cell secretory pathway, the VCAM-1 coding sequence was fused to signal peptide sequence of honeybee melittin. The resulting melittin-VCAM fusion was placed in correct orientation to the baculovirus polyhedrin promoter. Baculovirus transfer vector containing first 3-domain form VCAM-1 (pH10) was constructed by ligation of 0.9 kb fragment from AvrII/Klenow/BclI digests of pH7 into SalI/Klenow/BamHI digests of pMelBacB (Invitrogen). Recombinant baculovirus was generated by using Bac-N-Blue™ Transfection kit (Invitrogen) according to the manufacture's instruction. The recombinant virus was amplified by infection to High-Five™ insect cells for 5-6 days, and virus titer was determined by plaque assay.

High-Five™ insect cells were pelleted in a 225 ml conical tube by centrifugation at 1000 rpm for 5 min. After discarding the supernatant, the pellet was resuspended in 1.5×10⁹ pfu (MOI=5) of high-titer virus solution, followed by incubation for 1.5 hours at room temperature. The cells were pelleted again and washed once in fresh Express Five™ serum free medium. The cells were pelleted again and finally, resuspended in 200 ml of fresh Express Five TM medium, transferred to a 1,000 ml shaker flask, and incubated in a shaker at 27° C., 130 rpm, for 48 hours before the culture supernatant was collected. The purification of 3-domain form of VCAM-1 from the culture supernatant was performed by one-step anion exchange chromatography. Protein concentration was determined by using Coomassie protein assay reagent (Pierce) according to the manufacture's instruction.

Preparation of VCAM-1 Coated Microtiter Plates

Recombinant human VCAM-1 (extracellular domains 1-3) was dissolved at 1.0 μg/ml in PBS. Each well of the microtiter plates (Nalge Nunc International, Fluoronunc Cert, 437958) was coated with 100 μl of substrate or for background control with buffer alone for 15 hours at 4 C. After discarding the substrate solution, the wells were blocked using 150 μl per well of block solution (Kirkegaard Perry Laboratories, 50-61-01) for 90 minutes. The plate was washed with wash buffer containing 24 mM Tris-HCl (pH 7.4), 137 mM NaCl, 27 mM KCl and 2 mM MnCl₂ just before addition of the assay.

In Vitro Assay using Ramos Cells

Preparation of Fluorescence Labeled Ramos Cells:

Ramos cells (American Type Culture Collection, Clone CRL-1596) were cultured in RPMI 1640 medium (Nikken Bio Medical Laboratory, CM1101) supplemented with 10% fetal bovine serum (Hyclone, A-1119-L), 100 U/ml penicilin (Gibco BRL, 15140-122) and 100 μg/ml streptomycin (Gibco BRL, 15140-122) in a humidified incubator at 37° C. with 5% CO₂.

Ramos cells were incubated with phosphate balanced solution (PBS, Nissui, 05913) containing 25 μM of 5(-and 6)-carboxyfluorescein diacetate, succinimidyle ester (CFSE, Dojindo Laboratories, 345-06441) for 20 min at room temperature while gently swirling every 5 min. After centrifugation at 1000 rpm for 5 min, the cell pellet was resuspended with adhesion assay buffer at a cell density of 4×10⁶ cells/ml. The adhesion assay buffer was composed of 24 mM Tris-HCl (pH 7.4), 137 mM NaCl, 27 mM KCl, 4 mM glucose, 0.1% bovine serum albumin (BSA, Sigrna, A9647) and 2 mM MnCl₂.

Assay Procedure (Ramos Cells)

The assay solution containing each test compounds or 5 μg/ml anti-CD49d monoclonal antibody (Immunotech, 0764) was transferred to the VCAM-1 coated plates. The final concentration of each test compounds was 5 μM, 10 μM or various concentrations ranging from 0.0001 μM to 10 μM using a standard 5-point serial dilution. The assay solution containing the labeled Ramos cells was transferred to the VCAM-1 coated plates at a cell density of 2×10⁵ cells per well and incubated for 1 hour at 37 C. The non-adherent cells were removed by washing the plates 3 times with wash buffer. The adherent cells were broken by addition of 1% Triton X-100 (Nacalai Tesque, 355-01). Released CFSC was quantified fluorescence measurement in a fluorometer (Wallac, ARVO 1420 multilabel counter).

The adhesion of Ramos cells to VCAM-1 was analyzed by percent binding calculated by the formula:

100×(FTS−FBG)/(FTB−FBG)=% binding, where FTB is the total fluorescent intensity from VCAM-1 coated wells without test compound; FBG is the fluorescent intensity from wells with anti-CD49d monoclonal antibody and FTS is the fluorescent intensity from wells containing the test compound of this invention.

In Vitro Activity:

In the Jurkat-VCAM-1 assay (indicated as Jurkat-VCAM-1) and the Ramos-VCAM-1 (indicated as Ramos-VCAM-1) the observed IC₅₀ value ranges are indicated Table 4.
D>10μM≧C>2 μM≧B>0.5 μM≧A

TABLE 4

| Example No. | IC₅₀ |
|---|---|
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 49 | A |
| 50 | A |
| 51 | D |
| 52 | D |
| 53 | B |
| ^{53a} | A |
| 54 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | B |
| 79 | C |
| 80 | D |
| 81 | A |
| 82 | A |
| 83 | A |
| 84 | B |
| 85 | A |
| 86 | A |
| 87 | A |
| 88 | A |
| 89 | A |
| 90 | A |
| 91 | B |
| 92 | B |

1. A compound of the general formula (I), 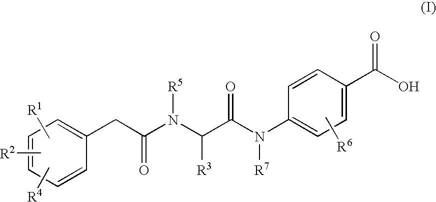
wherein
R¹ represents hydrogen, C₁-C₄-alkyl, trifluormethyl, trifluormethoxy, phenyl, —OR¹⁻², —SR¹⁻², NR¹⁻³R¹⁻⁴, —C(O)R¹⁻², S(O)R¹⁻², —SO₂R¹⁻², —CO₂R¹⁻², —OC(O)R¹⁻², —C(O)NR¹⁻³R¹⁻⁴, —NR¹⁻²C(O)R¹⁻², —SO₂NR¹⁻³R¹⁻⁴, —NR¹⁻² SO₂R¹⁻², —NR¹⁻²C(O)NR¹⁻³R¹⁻⁴, —NR¹⁻²C(O)OR¹⁻⁴, —OC(O)NR¹⁻³R¹⁻⁴, halogen, cyano, nitro or amino,
wherein R¹⁻² represents hydrogen or C₁-C₄-alkyl,
wherein R¹⁻³ represents hydrogen or C₁-C₄-alkyl,
R¹⁻⁴ represents hydrogen, C₁-C₄-alkyl, C₃-C₆-cycloalkyl, C₆- or C₁₀-aryl, heteroaryl or a heterocycle,
wherein R¹⁻⁴ can optionally be substituted by 1 to 2 substituents selected from the group consisting of C₁-C₄-alkyl, phenyl, C₃-C₇-cycloalkyl, C₁-C₄-alkyloxy, halogen, nitro, and cyano,
R² represents hydrogen or halogen, or
R¹ and R² together form a 4-7-membered ring, which includes the carbon atoms to which R¹ and R² are bonded and which contains up to 2 additional heteroatoms selected from the group consisting of oxygen, nitrogen of and sulfur and which contains up to 2 double bonds,
wherein the ring formed by R¹ and R² can optionally be substituted by —NH—C₆— or C₁₀-aryl, —NH-heterocyclyl or —NH-heteroaryl,
wherein C₆- or C₁₀-aryl can optionally be substituted by 1 to 2 substituents halogen, C₁-C₄-alkyl or C₁-C₄-alkoxy,
R³ represents hydrogen, C₁-C₁₀-alkyl, C₂-C₁₀-alkenyl, C₂-C₁₀-alkynyl, —(CH₂)ₘ—C₆— or C₁₀-aryl, —(CH₂)ₘ—C₃-C₇-cycloalkyl, —(CH₂)ₘ-heterocyclyl or —(CH₂)ₘ-heteroaryl,
wherein m represents an integer of zero to six,
wherein R³ can optionally be substituted by 1 to 3 radicals R³⁻¹,
wherein R³⁻¹ represents trifluormethyl, trifluormethoxy, —OR³⁻², —NR³⁻³R³⁻⁴, —C(O)R³⁻², halogen, cyano, nitro, oxo, C₆- or C₁₀-aryl, heterocyclyl, or heteroaryl,
wherein R³⁻² represents hydrogen, C₁-C₄-alkyl, C₃-C₆-cycloalkyl, or C₆- or C₁₀-aryl,
and wherein R³⁻³ and R³⁻⁴ are identical or different and represent hydrogen or C₁-C₄-alkyl,
R⁴ represents hydrogen, halogen, C₁-C₄-alkyl, C₁-C₄-alkoxy, cyano, amino or nitro,
R⁵ represents hydrogen, C₁-C₁₀-alkyl, C₂-C₁₀-alkenyl, C₂-C₁₀-alkynyl, —(CH₂)ₙ—C₆— or C₁₀-aryl, —(CH₂)ₙ—C₃-C₇-cycloalkyl, —(CH₂)ₙ-heterocyclyl, or —(CH₂)ₙ-heteroaryl,
wherein n represents an integer of zero to six,
wherein R⁵ can optionally be substituted by 1 to 3 radicals R⁵⁻¹,
wherein R⁵⁻¹ represents C₁-C₄ alkyl, trifluormethyl, trifluormethoxy, —OR⁵⁻², —NR⁵⁻³R⁵⁻⁴, —C(O)R⁵⁻², halogen, cyano, nitro, oxo, C₆- or C₁₀-aryl, heterocyclyl, or heteroaryl,
wherein R⁵⁻² represents hydrogen, C₁-C₄-alkyl, C₃-C₆-cycloalkyl, C₆- or C₁₀-aryl or halogenated C₆- or C₁₀-aryl,
and wherein R⁵⁻³ and R⁵⁻⁴ are identical or different and represent hydrogen or C₁-C₄-alkyl, or
R³ and R⁵ together form a 4-7-membered heterocyclic ring, which includes the nitrogen atom to which R⁵ is bonded and the carbon atom to which R³ is bonded and which contains up to 2 additional heteroatoms selected from the group oxygen, nitrogen and sulfur and which contains up to 2 double bonds,
R⁶ represents hydrogen, C₁-C₄ alkyl, —OR⁶⁻¹, —NR⁶⁻²R⁶⁻³, —C(O)R⁶⁻¹, C₆-aryl, heterocyclyl, heteroaryl, halogen, cyano, nitro, hydroxy, amino, trifluoromethyl, or trifluoromethoxy,
wherein R⁶⁻¹ represents hydrogen, C₁-C₄-alkyl, C₃-C₆-cycloalkyl or C₆-aryl,
wherein R⁶⁻² and R⁶⁻³ are identical or different and represent hydrogen, C₁-C₄-alkyl, C₃-C₆-cycloalkyl or C₆-aryl,
and wherein R⁶, R⁶⁻¹, R⁶⁻² and R⁶⁻³ can optionally be substituted by 1 to 2 radicals R⁶⁻⁴,
wherein R⁶⁻⁴ represents trifluoromethyl, trifluoromethoxy, halogen, cyano, nitro, hydroxy, amino and or oxo
R⁷ represents hydrogen or C₁-C₄ alkyl,
or R⁷ and R³ together with the carbon atoms to which they are bonded form a cycloalkyl ring,
X represents oxygen or two hydrogen atoms,
or a pharmaceutically acceptable salt thereof.

2. The compound according to claim 1, wherein
R¹ represents —NR¹⁻²C(O)NR¹⁻³R¹⁻⁴,
wherein R¹⁻² represents hydrogen,
wherein R¹⁻³ represents hydrogen,
wherein R¹⁻⁴ represents C₆- or C₁₀-aryl or pyridyl,
wherein R¹⁻⁴ can optionally be substituted by 1 to 2 substituents C₁-C₄-alkyl, C₁-C₄-alkoxy or halogen,
R represents hydrogen, halogen, C₁-C₄-alkyl or C₁-C₄-alkoxy, or
R¹ and R² together form a 4-6-membered heterocyclic or heteroaromatic ring, which includes the carbon atoms to which R¹ and R² are bonded and which contains 1 or 2 additional heteroatoms selected from the group consisting of oxygen and nitrogen and which contains 1 or 2 double bonds,
wherein the ring formed by R¹ and R² can optionally be substituted by —NH—C₆— or C₁₀-aryl,
wherein C₆- or C₁₀-aryl can optionally be substituted by 1 to 2 substituents halogen, C₁-C₄-alkyl or C₁-C₄-alkoxy,
R³ represents hydrogen, C₁-C₁₀-alkyl, —(CH₂)ₘ—C₆— or C₁₀-aryl, —(CH₂)ₘ—C₃-C₇-cycloalkyl, —(CH₂)ₘ-heterocyclyl, or —(CH₂)ₘ-heteroaryl,
wherein m represents an integer of one to four,
wherein R³ can optionally be substituted by 1 to 2 radicals R³⁻¹,
wherein R³⁻¹ represents —OR³⁻², —NR³⁻³R³⁻⁴, —C(O)R³⁻², halogen, cyano, nitro, oxo, C₆- or C₁₀-aryl, heterocyclyl, or heteroaryl,
wherein R³⁻² represents hydrogen or C₁-C₄-alkyl,
and wherein R³⁻³ and R³⁻⁴ are identical or different and represent hydrogen or C₁-C₄-alkyl,
R⁴ represents hydrogen, halogen, C₁-C₄-alkyl or C₁-C₄-alkoxy,
R⁵ represents hydrogen, C₁-C₁₀-alkyl, —(CH₂)ₙ—C₆— or C₁₀-aryl, —(CH₂)ₙ—C₃-C₇-cycloalkyl, —(CH₂)ₙ-heterocyclyl, or —(CH₂)ₙ-heteroaryl,
wherein n represents an integer of one to three,
wherein R⁵ can optionally be substituted by 1 to 2 radicals R⁵⁻¹,
wherein R⁵⁻¹ represents C₁-C₄-alkyl, —OR⁵⁻², —NR⁵⁻³R⁵⁻⁴, —C(O)R⁵⁻², halogen, cyano, nitro, oxo, C₆- or C₁₀-aryl, heterocyclyl, or heteroaryl,
wherein R⁵⁻² represents hydrogen or C₁-C₄-alkyl,
and wherein R⁵⁻³ and R⁵⁻⁴ are identical or different and represent hydrogen or C₁-C₄-alkyl,
R⁶ represents hydrogen,
R⁷ represents hydrogen or C₁-C₄ alkyl,
or R⁷ and R³ together with the carbon atoms to which they are bonded form a cycloalkyl ring,
X represents oxygen or two hydrogen atoms,
R⁷ represents hydrogen,
X represents oxygen,
or a pharmaceutically acceptable salt thereof.

3. The compound according to claim 1, wherein
R¹ represents —NR¹⁻² C(O)NR¹⁻³R¹⁻⁴,
wherein R¹⁻² represents hydrogen,
wherein R¹⁻³ represents hydrogen,
wherein R¹⁻⁴ represents C₆-aryl,
wherein R¹⁻⁴ is substituted by 1 to 2 substituents C₁-C₄-alkyl,
R² represents hydrogen, or
R¹ and R² together form a 5-membered heterocyclic or heteroaromatic ring, which includes the carbon atoms to which R¹ and R² are bonded and which contains 1 or 2 additional heteroatoms selected from the group consisting of oxygen and nitrogen and which contains 1 or 2 double bonds,
wherein the ring formed by R¹ and R² can optionally be substituted by —NH—C₆ aryl,
wherein C₆- or C₁₀-aryl can optionally be substituted by 1 to 2 substituents halogen, C₁-C₄-alkyl or C₁-C₄-alkoxy,
R³ represents hydrogen, C₁-C₁₀-alkyl, —(CH₂)ₘ—C₆-aryl, —(CH₂)ₘ—C₃-C₇-cycloalkyl, —(CH₂)ₘ-heterocyclyl, or —(CH₂)ₘ-heteroaryl,
wherein m represents an integer of one or two,
wherein R³ can optionally be substituted by 1 to 2 radicals R³⁻¹,
wherein R³⁻¹ represents —OR³⁻², —NR³⁻³R³⁻⁴, —C(O)R³⁻², halogen, oxo, C₆- or C₁₀-aryl, heterocyclyl, or heteroaryl,
wherein R³⁻² represents hydrogen or C₁-C₄-alkyl,
and wherein R³⁻³ and R³⁻⁴ are identical or different and represent hydrogen or C₁-C₄-alkyl,
R⁴ represents hydrogen, halogen, C₁-C₄-alkyl or C₁-C₄-alkoxy,
R⁵ represents hydrogen, C₁-C₁₀-alkyl, —(CH₂)ₙ—C₆-aryl, —(CH₂)ₙ—C₃-C₇-cycloalkyl, —(CH₂)ₙ-heterocyclyl, or —(CH₂)ₙ-heteroaryl,
wherein n represents an integer of one to three,
wherein R⁵ can optionally be substituted by 1 to 2 radicals R⁵⁻¹,
wherein R⁵⁻¹ represents C₁-C₄-alkyl, —OR⁵⁻², —NR⁵⁻³R⁵⁻⁴, —C(O)R⁵⁻², halogen, cyano, nitro, oxo, C₆- or C₁₀-aryl, heterocyclyl, or heteroaryl,
wherein R⁵⁻² represents hydrogen or C₁-C₄-alkyl,
and wherein R⁵⁻³ and R⁵⁻⁴ are identical or different and represent hydrogen or C₁-C₄-alkyl,
R⁶ represents hydrogen,
R⁷ represents hydrogen,
X represents oxygen,
or a pharmaceutically acceptable salt thereof.

4. The compound according to claim 1, wherein R¹ represents a group of the formula

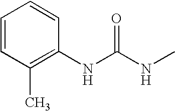

5. The compound according to claim 1, wherein the group of the formula 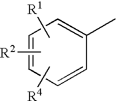
represents a group of the formula

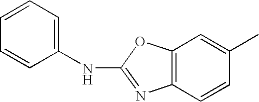

6. The compound according to claim 1, wherein the group of the formula 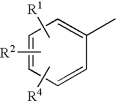
represents a group of the formula

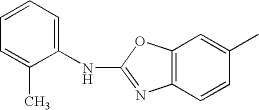

7. The compound according to claim 1, wherein
R³ represents hydrogen.

8. The compound according to claim 1, wherein the compound is selected from the following group:
4-[(N²-{[4-({[(2L-methylphenyl)amino]carbonyl}amino)phenyl]acetyl}-D-lysyl)-amino]benzoic acid trifluoroacetate,
4-[(N-[3-(dimethylamino)propyl]-N-{[4-({[(2-methylphenyl)amino]carbon-yl}amino)phenyl]acetyl}glycyl)amino]benzoic acid,
4-[(N-(4-aminobutyl)-N-{[4-({[(2-methylphenyl)amino]carbonyl}amino)-phenyl]acetyl}glycyl)amino]benzoic acid,
4-({N-{[4-({[(2-methylphenyl)amino]carbonyl}amino)phenyl]acetyl}-N-[3-(1-pyrrolidinyl)propyl]glycyl}amino)benzoic acid,
4-[(N-[(1-ethyl-2-pyrrolidinyl)methyl]-N-{[4-({[(2-methylphenyl)amino]-carbonyl}amino)phenyl]acetyl}glycyl)amino]benzoic acid,
4-({N-{[4-({[(2-methylphenyl)amino]carbonyl}amino)phenyl]acetyl}-N-[3-(4-phenyl-1-piperazinyl)propyl]glycyl}amino)benzoic acid,
4-{[N-{[4-({[(2-methylphenyl)amino]carbonyl}amino)phenyl]acetyl}-N-(tetrahydro-2-furanylmethyl)glycyl]amino}benzoic acid,
4-{[N-{[4-({[(2-methylphenyl)amino]carbonyl}amino)phenyl]acetyl}-N-(4-piperidinylmethyl)glycyl]amino}benzoic acid,
4-[(N-(3-amino-2,2-dimethylpropyl)-N-{[4-({[(2-methylphenyl)amino]-carbonyl}amino)phenyl]acetyl}glycyl)amino]benzoic acid,
4-({N-{[4-({[(2-methylphenyl)amino]carbonyl}amino)phenyl]acetyl}-N-[2-(1-pyrrolidinyl)ethyl]glycyl}amino)benzoic acid,
4-[(N-{[4-({[(2-methylphenyl)amino]carbonyl}amino)phenyl]acetyl}-N-propylglycyl)amino]benzoic acid,
4-({N-{[4-({[(2-methylphenyl)amino]carbonyl}amino)phenyl]acetyl}-N-[3-(2-oxo-1-pyrrolidinyl)propyl]glycyl}amino)benzoic acid,
4-[(N-(2-methoxyethyl)-N-{[4-({[(2-methylphenyl)amino]carbonyl}amino)-phenyl]acetyl}glycyl)amino]benzoic acid,
4-({N-{[4-({[(2-methylphenyl)amino]carbonyl}amino)phenyl]acetyl}-N-[3-(4-morpholinyl)propyl]glycyl}amino)benzoic acid,
4-{[N {[4-({[(2-methylphenyl)amino]carbonyl}amino)phenyl]acetyl}-N-(3-pyridinylmethyl)glycyl]amino}benzoic acid,
4-{[N-{[4-({[(2-methylphenyl)amino]carbonyl}amino)phenyl]acetyl}-N-(2-pyridinylmethyl)glycyl]amino}benzoic acid,
4-{[N-{[4-({[(2-methylphenyl)amino]carbonyl}amino)phenyl]acetyl}-N-(4-yridinylmethyl)glycyl]amino}benzoic acid,
4-[(N-[2-(1H-imidazol-4-yl)ethyl]-N-{[4-({[(2-methylphenyl)amino]carbon-yl}amino)phenyl]acetyl}glycyl)amino]benzoic acid,
4-({N-{[4-({[(2-methylphenyl)amino]carbonyl}amino)phenyl]acetyl}-N-[2-(2-pyridinyl)ethyl]glycyl}amino)benzoic acid,
4-({N-[(2-anilino-1,3-benzoxazol-6-yl)acetyl]glycyl}amino)benzoic acid,
4-{[N-[(2-anilino-1,3-benzoxazol-6-yl)acetyl]-N-(2-phenylethyl)glycyl]-amino}benzoic acid,
4-({N-[(2-anilino-1,3-benzoxazol-6-yl)acetyl]-N-[2-(2-pyridinyl)ethyl]-glycyl}amino)benzoic acid,
4-({N-[(2-anilino-1,3-benzoxazol-6-yl)acetyl]-N-[2-(3,5-dimethoxyphenyl)-ethyl]glycyl}amino)benzoic acid,
4-{[N-({2-[(2-methylphenyl)amino]-1,3-benzoxazol-6-yl}acetyl)glycyl]amino}-benzoic acid,
4-{[N-({2-[(2-methylphenyl)amino]-1,3-benzoxazol-6-yl}acetyl)-N-(2-phenylethyl)glycyl]amino}benzoic acid,
4-({N-({2-[(2-methylphenyl)amino]-1,3-benzoxazol-6-yl}acetyl)-N-[2-(2-pyridinyl)ethyl]glycyl}amino)benzoic acid,
4-[(N-[2-(3-methoxyphenyl)ethyl]-N-{[4-({[(2-methylphenyl)amino]-carbonyl}amino)phenyl]acetyl}glycyl)amino]benzoic acid,
4-[(N-benzyl-N-{[4-({[(2-methylphenyl)amino]carbonyl}amino)phenyl]-acetyl}glycyl)amino]benzoic acid,
4-({N-[(2-anilino-1,3-benzoxazol-6-yl)acetyl]-N-[2-(3-methoxyphenyl)-ethyl]glycyl}amino)benzoic acid,
4-[(N-{[4-({[(2-methylphenyl)amino]carbonyl}amino)phenyl]acetyl}-L-phenylalanyl)amino]benzoic acid,
4-({N-[(2-anilino-1,3-benzoxazol-6-yl)acetyl]-L-phenylalanyl}amino)benzoic acid,
4-[(4-bromo-N-{[4-({[(2-methylphenyl)amino]carbonyl}amino)phenyl]acetyl}-L-phenylalanyl)amino]benzoic acid,
4-[(N-{[4-({[(2-methylphenyl)amino]carbonyl}amino)phenyl]acetyl}glycyl)amino]benzoic acid
4-{[(2S)-4-amino-2-({[4-({[(2-methylphenyl)amino]carbonyl}amino)phenyl]acetyl}amino)butanoyl]amino}benzoic acid
4-[(N²-{[4-({[(2-methylphenyl)amino]carbonyl}amino)phenyl]acetyl}-L-ornithyl)amino]benzoic acid
4-[(N-{[4-({[(2-methylphenyl)amino]carbonyl}amino)phenyl]acetyl}-L--aspartyl)amino]benzoic acid
4-[(N-{[4-({[(2-methylphenyl)amino]carbonyl}amino)phenyl]acetyl}-L-tryptophyl)amino]benzoic acid
4-{[N-{[4-({[(2-methylphenyl)amino]carbonyl}amino)phenyl]acetyl}-3-(4-pyridinyl)-L-alanyl]amino}benzoic acid
4-{[N-{[4-({[(2-methylphenyl)amino]carbonyl}amino)phenyl]acetyl}-3-(3-pyridinyl)-L-alanyl]amino}benzoic acid
4-{[N-{[4-({[(2-methylphenyl)amino]carbonyl}amino)phenyl]acetyl}-3-(1,3-thiazol-4-yl)-L-alanyl]amino}benzoic acid
4-[(N-{[4-({[(2-methylphenyl)amino]carbonyl}amino)phenyl]acetyl}-L-histidyl)amino]benzoic acid
4-{[(1-{[4-({[(2-methylphenyl)amino]carbonyl}amino)phenyl]acetyl}-2-piperazinyl)carbonyl]amino}benzoic acid
4-[3-({[4-({[(2-methylphenyl)amino]carbonyl}amino)phenyl]acetyl}amino)-1-piperidinyl]benzoic acid
4-[3-({[4-({[(2-methylphenyl)amino]carbonyl}amino)phenyl]acetyl}amino)-1-pyrrolidinyl]benzoic acid
4-[isobutyl(N-{[4-({[(2-methylphenyl)amino]carbonyl}amino)phenyl]acetyl}glycyl)amino]benzoic acid
4-[isobutyl(N-(3-methoxypropyl)-N-{[4-({[(2-methylphenyl)amino]carbonyl}amino)phenyl]acetyl}glycyl)amino]benzoic acid and
4-[(N-(3-methoxypropyl)-N-{[4-({[(2-methylphenyl)amino]carbonyl}amino)phenyl]acetyl}glycyl)(methyl)amino]benzoic acid.

9. (Cancelled)

10. A method for the treatment or the prevention of a condition mediated by integrins comprising administering an effective amount of a compound of claim 1.

11. The method of claim 10 wherein said condition mediated by integrins is selected from the group consisting of atherosclerosis, asthma, chronic obstructive pulmonary disease (COPD), allergies, diabetes, inflammatory bowel disease, multiple sclerosis, myocardial ischemia, rheumatoid arthritis, transplant rejection and other inflammatory, autoimmune and immune disorders.

12. A pharmaceutical composition, comprising a compound according to claim 1 and a pharmaceutically acceptable carrier.

13. (Cancelled)
